(19) United States
(12) Patent Application Publication
(10) Pub. No.: US 2013/0067609 A1
(43) Pub. Date: Mar. 14, 2013

(54) Mammalian Genes Involved in Tularemia and Other Infections

(71) Applicants: Donald Rubin, Nashville, TN (US); Robert Gilmore, Fort Collins, CO (US); Thomas Hodge, Athens, GA (US); Natalie McDonald, Atlanta, GA (US)

(72) Inventors: Donald Rubin, Nashville, TN (US); Robert Gilmore, Fort Collins, CO (US); Thomas Hodge, Athens, GA (US); Natalie McDonald, Atlanta, GA (US)

(21) Appl. No.: 13/377,815

(22) PCT Filed: Jun. 14, 2010
(86) PCT No.: PCT/US10/38515

§ 371 Date: Oct. 16, 2012

Related U.S. Application Data
(60) Provisional application No. 61/268,471, filed on Jun. 12, 2009.

(51) Int. Cl. A61K 31/713; A01K 67/027; C12N 5/071

(52) U.S. Cl. 800/14; 435/375; 514/44 A

(57) ABSTRACT

The present invention relates to nucleic acid sequences and cellular proteins encoded by these sequences that are involved in infection or are otherwise associated with the life cycle of one or more pathogens.

This application claims the benefit of U.S. Application No. 61/268,471, filed on Jun. 12, 2009, which is hereby incorporated in its entirety by this reference.

### FIELD OF THE INVENTION

The present invention relates to nucleic acid sequences and cellular proteins encoded by these sequences that are involved in infection or are otherwise associated with the life cycle of one or more pathogens, such as a virus, a bacteria, a fungus or a parasite. The invention also relates to modulators of nucleic acid sequences and cellular proteins encoded by these sequences that are involved in infection or are otherwise associated with the life cycle of a pathogen.

### BACKGROUND

Infectious diseases affect the health of people and animals around the world, causing serious illness and death. Black Plague devastated the human population in Europe during the middle ages. Pandemic flu killed millions of people in the 20^{th} century and is a threat to reemerge.

Some of the most feared, widespread, and devastating human diseases are caused by viruses that interfere with normal cellular processes. These include influenza, poliomyelitis, smallpox, Ebola, yellow fever, measles and AIDS, to name a few. Viruses are also responsible for many cases of human disease including encephalitis, meningitis, pneumonia, hepatitis and cervical cancer, warts and the common cold. Furthermore, viruses causing respiratory infections, and diarrhea in young children lead to millions of deaths each year in less-developed countries. Also, a number of newly emerging human diseases such as SARS are caused by viruses. In addition, the threat of a bioterrorist designed pathogen is ever present.

While vaccines have been effective to prevent certain viral infections, relatively few vaccines are available or wholly effective, have inherent risks and tend to be specific for particular conditions. Vaccines are of limited value against rapidly mutating viruses and cannot anticipate emerging viruses or new bioterrorist designed viruses. Currently there is no good answer to these threats.

Traditional treatments for viral infection include pharmaceuticals aimed at specific virus derived proteins, such as HIV protease or reverse transcriptase, or the administration of recombinant (cloned) immune modulators (host derived), such as the interferons. However, the vast majority of viruses lack an effective drug. Those drugs that exist have several limitations and drawbacks that including limited effectiveness, toxicity, and high rates of viral mutations which render antiviral pharmaceuticals ineffective. Thus, an urgent need exists for alternative treatments for viruses and other infectious diseases, and methods of identifying new drugs to combat these threats.

### SUMMARY OF THE INVENTION

The present invention provides EIF5, HNRNPU, BST1, FBXL5, FUT4 and SNORA28 nucleic acid sequences and proteins encoded by these sequences that are involved in infection by one or more pathogens such as a virus, a parasite, a bacteria or a fungus, or are otherwise associated with the life cycle of a pathogen. Also provided are methods of decreasing infection in a cell by a pathogen comprising decreasing expression or activity of one or more of EIF5, HNRNPU, BST1, FBXL5, FUT4 and SNORA28. Also provided are methods of decreasing infection by a pathogen in a subject by administering an agent that decreases the expression and/or activity of EIF5, HNRNPU, BST1, FBXL5, FUT4 or SNORA28. Further provided are methods of identifying an agent that decreases infection by a pathogen.

### DETAILED DESCRIPTION OF THE INVENTION

The present invention may be understood more readily by reference to the following detailed description of preferred embodiments of the invention and the Examples included therein.

Before the present compounds, compositions and/or methods are disclosed and described, it is to be understood that this invention is not limited to specific nucleic acids, specific polypeptides, or to particular methods, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally obtained prior to treatment” means obtained before treatment, after treatment, or not at all.

As used throughout, by “subject” is meant an individual. Preferably, the subject is a mammal such as a primate, and, more preferably, a human. Non-human primates include marmosets, monkeys, chimpanzees, gorillas, orangutans, and gibbons, to name a few. The term “subject” includes domesticated animals, such as cats, dogs, etc., livestock (for example, cattle, horses, pigs, sheep, goats, etc.), laboratory animals (for example, ferret, chinchilla, mouse, rabbit, rat, gerbil, guinea pig, etc.) and avian species (for example, chickens, turkeys, ducks, pheasants, pigeons, doves, parrots, cockatoos, geese, etc.). The subjects of the present invention can also include, but are not limited to fish (for example, zebrafish, goldfish, tilapia, salmon and trout), amphibians and reptiles.

EIF5, HNRNPU, BST1, FBXL5, FUT4 and SNORA28, host genes involved in infection, were identified using gene trap methods that were designed to identify host genes that are necessary for infection or growth, but nonessential for cellular survival. These gene trap methods are set forth in the Examples as well as in U.S. Pat. No. 6,448,000 and U.S. Pat. No. 6,777,177. U.S. Pat. Nos. 6,448,000 and 6,777,177 and are both incorporated herein in their entireties by this reference.

As used herein, a gene “nonessential for cellular survival” means a gene for which disruption of one or both alleles results in a cell viable for at least a period of time which allows viral replication to be decreased or inhibited in a cell. Such a decrease can be utilized for preventative or therapeutic uses or used in research. A gene necessary for pathogenic infection or growth means the gene product of this gene, either protein or RNA, secreted or not, is necessary, either directly or indirectly in some way for the pathogen to grow. As utilized throughout, “gene product” is the RNA or protein resulting from the expression of a gene.

The nucleic acids set forth herein and their encoded proteins can be involved in all phases of viral life cycles including, but not limited to, viral attachment to cellular receptors, viral infection, viral entry, internalization, disassembly of the virus, viral replication, genomic integration of viral sequences, transcription of viral RNA, translation of viral mRNA, transcription of cellular proteins, translation of cellular proteins, trafficking, proteolytic cleavage of viral proteins or cellular proteins, assembly of viral particles, budding, cell lysis and egress of virus from the cells.

Although EIF5, HNRNPU, BST1, FBXL5, FUT4 and SNORA28 were identified as cellular genes involved in franciscella tularensis (tularemia) infection, as discussed throughout, the present invention is not limited to tularemia. Therefore, any of these nucleic acid sequence and the proteins encoded by these sequences can be involved in infection by any infectious pathogen such as a bacteria, a virus, a fungus or a parasite which includes involvement in any phase, of the infectious pathogen's life cycle.

EIF5, HNRNPU, BST1, FBXL5, FUT4 and SNORA28 are set forth in Table 1 as genes involved in infection. Additional identifying information for each of these genes is also set forth in Table 1. As utilized herein, when referring to any of the genes in this table, for example, and not to be limiting, EIF5, this includes any EIF5 gene, nucleic acid (DNA or RNA) or protein from any organism that retains at least one activity of EIF5 and can function as an EIF5 nucleic acid or protein utilized by a pathogen. For example, the nucleic acid or protein sequence can be from or in a cell in a human, a non-human primate, a mouse, a rat, a cat, a dog, a chimpanzee, a horse, a cow, a pig, a sheep, a guinea pig, a rabbit, a zebrafish, a chicken, to name a few.

As used herein, a gene is a nucleic acid sequence that encodes a polypeptide under the control of a regulatory sequence, such as a promoter or operator. The coding sequence of the gene is the portion transcribed and translated into a polypeptide (in vivo, in vitro or in situ) when placed under the control of an appropriate regulatory sequence. The boundaries of the coding sequence can be determined by a start codon at the 5′ (amino) terminus and a stop codon at the 3′ (carboxyl) terminus. If the coding sequence is intended to be expressed in a eukaryotic cell, a polyadenylation signal and transcription termination sequence can be included 3′ to the coding sequence. SNORA28 listed in Table 1 encodes RNA molecules and does not encode a polypeptide. Therefore, this gene encodes non-protein coding RNA sequences.

Transcriptional and translational control sequences include, but are not limited to, DNA regulatory sequences such as promoters, enhancers, and terminators that provide for the expression of the coding sequence, such as expression in a host cell. A polyadenylation signal is an exemplary eukaryotic control sequence. A promoter is a regulatory region capable of binding RNA polymerase and initiating transcription of a downstream (3′ direction) coding sequence. Additionally, a gene can include a signal sequence at the beginning of the coding sequence of a protein to be secreted or expressed on the surface of a cell. This sequence can encode a signal peptide, N-terminal to the mature polypeptide, which directs the host cell to translocate the polypeptide.

Table 1 (column 2) provides one or more aliases for each of the genes set forth herein. Therefore, it is clear that when referring to a gene, this also includes known alias(es) and any aliases attributed to the genes listed in Table 1 in the future. The proteins encoded by the genes, if available, are also listed in column 3 of Table 1. In addition to the function of being involved in pathogenic infection as provided herein, a function of the proteins is also provided, if available, in column 4 of Table 1. The chromosomal location of the gene in the human genome (column 5) is also set forth. Thus, the present invention identifies a genomic loci of genes associated with viral infection. By identifying the gene and its location in the genome, the invention provides both the gene and its product(s) as targets for therapies such as antiviral, antibacterial, antifungal and antiparasitic therapies, to name a few.

Also provided in Table 1 are the GenBank Accession Nos. for the human mRNA sequences (column 6) and the GenBank Accession Nos. for the human protein sequences (column 7), if available. It is understood that in any coding sequence, a T can be replaced with a U to obtain an RNA sequence for each gene. A SEQ ID NO: is provided in parentheses after the GenBank Accession number for each mRNA and protein sequence set forth in Table 1. The nucleic acid sequences and protein sequences provided under the GenBank Accession Nos. mentioned herein are hereby incorporated in their entireties by this reference. One of skill in the art would know that the nucleotide sequences provided under the GenBank Accession Nos. set forth herein can be readily obtained from the National Center for Biotechnology Information at the National Library of Medicine (http://www.ncbi.nlm.nih.gov/entrez/query.fcgi?db=nucleotide). Similarly, the protein sequences set forth herein can be readily obtained from the National Center for Biotechnology Information at the National Library of Medicine (http://www.ncbi.nlm.nih.gov/entrez/query.fcgi?db=protein). The nucleic acid sequences and protein sequences provided under the GenBank Accession Nos. mentioned herein are hereby incorporated in their entireties by this reference.

These examples are not meant to be limiting as one of skill in the art would know how to obtain additional sequences for the genes and gene products listed in Table 1 from other species by accessing GenBank (Benson et al. Nucleic Acids Res. 2004 January 1; 32(Database issue); D23-D26), the EMBL Database (Stoesser et al., (2000) Nucleic Acids Res., 28, 19-23) or other sequence databases. One of skill in the art would also know how to align the sequences disclosed herein with sequences from other species in order to determine similarities and differences between the sequences set forth in Table 1 and related sequences, for example, by utilizing BLAST. As set forth herein, a nucleic acid sequence for any of the genes set forth in Table 1 can be a full-length wild-type (or native) sequence, a genomic sequence, a variant (for example, an allelic variant or a splice variant), a nucleic acid fragment, a homolog or a fusion sequence that retains the activity of the gene utilized by the pathogen or its encoded gene product. For example, EIF5 activity includes but it not limited eukaryotic translation initiation as well as the ability to function as a cellular nucleic acid or protein involved in infection.

Further provided are the Entrez Gene numbers for the human genes (column 8). The information provided under the Entrez Gene numbers listed in Table 1 is also hereby incorporated entirely by this reference. One of skill in the art can readily obtain this information from the National Center for Biotechnology Information at the National Library of Medicine (http://www.ncbi.nlm.nih.gov/entrez/query.fcgi?db=gene). By accessing Entrez Gene, one of skill in the art can readily obtain additional information about every gene listed in Table 1, such as the genomic location of the gene, a summary of the properties of the protein encoded by the gene, information on homologs of the gene as well as numerous reference sequences, such as the genomic, mRNA and protein sequences for each gene. Thus, in addition to the sequences set forth under the GenBank Accession Nos. in Table 1, one of skill in the art can readily obtain additional sequences, such as genomic, mRNA and protein sequences by accessing additional information available under the Entrez Gene number provided for each gene. Thus, all of the information readily obtained from the Entrez Gene Nos. set forth herein is also hereby incorporated by reference in its entirety.

| | | | | Human | Human GenBank | Human GenBank | |
|---|---|---|---|---|---|---|---|
| | | | | Chromo- | Accession No. | Accession No. | Entrez |
| | | | | somal | for mRNA | for protein | Gene |
| Gene | Alias | Definition | Known Cellular Functions | Location | (SEQ ID NO:) | (SEQ ID NO:) | No. |
| EIF5 | EIF-5A | eukaryotic | Involved in translation | 14q32.32 | NM_001969.3 (1) | NP_001960.2 (2) | 1983 |
| | | translation | | | NM_183004.3 (3) | NP_892116.2 (4) | |
| | | initiation factor 5 | | | | | |
| HNRNPU | SAF-A; U21.1; | heterogeneous | hnRNPs are RNA binding | 1q44 | NM_004501.3 (5) | NP_004492.2 (6) | 3192 |
| | HNRPU | nuclear | proteins and they form | | NM_031844.2 (7) | NP_114032.2 (8) | |
| | | ribonucleoprotein U | complexes with heterogeneous | | | | |
| | | (scaffold attachment | nuclear RNA (hnRNA). These | | | | |
| | | factor A) | proteins are associated with | | | | |
| | | | pre-mRNAs in the nucleus and | | | | |
| | | | appear to influence pre-mRNA | | | | |
| | | | processing and other aspects of | | | | |
| | | | mRNA metabolism and | | | | |
| | | | transport. | | | | |
| BST1 | CD157 | bone marrow | A stromal cell line-derived | 4p15 | NM_004334.2 (9) | NP_004325.2 (10) | 683 |
| | | stromal cell antigen | glycosylphosphatidylinositol- | | | | |
| | | 1 | anchored molecule that | | | | |
| | | | facilitates pre-B-cell growth. | | | | |
| FBXL5 | FBL4; FBL5; | F-box and leucine- | F-box proteins constitute one of | 4p15.33 | NM_012161.2 (11) | NP_036293.1 (12) | 26234 |
| | FLR1 | rich repeat protein 5 | the four subunits of ubiquitin | | NM_033535.2 (13) | NP_277077.1 (14) | |
| | | | protein ligase complex called | | | | |
| | | | SCFs (SKP1-cullin-F-box), | | | | |
| | | | which function in | | | | |
| | | | phosphorylation-dependent | | | | |
| | | | ubiquitination. | | | | |
| FUT4 | CD15; ELFT; | fucosyltransferase 4 | Transfers fucose to N- | 11q21 | NM_002033.3 (15) | NP_002024.1 (16) | 2526 |
| | FCT3A; | (alpha (1,3) | acetyllactosamine | | | | |
| | FUTIV; | fucosyltransferase, | polysaccharides to generate | | | | |
| | FUC-TIV | myeloid-specific) | fucosylated carbohydrate | | | | |
| | | | structures. | | | | |
| SNORA28 | ACA28 | small nucleolar | Member of the H/ACA class of | 14q32.32 | NR_002964.1 (17) | Not applicable | 677811 |
| | | RNA, H/ACA box | small nucleolar RNA that guide | | | | |
| | | 28 | the sites of modification of | | | | |
| | | | uridines to pseudouridines. | | | | |

As used herein, the term “nucleic acid” refers to single or multiple stranded molecules, which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for the methods. These techniques can be used to alter the coding sequence without altering the amino acid sequence that is encoded.

The sequences contemplated herein include full-length wild-type (or native) sequences, as well as allelic variants, variants, fragments, homologs or fusion sequences that retain the ability to function as the cellular nucleic acid or protein involved in viral infection. In certain examples, a protein or nucleic acid sequence has at least 50% sequence identity, for example at least 50%, 55%, 60%, 65%, 70%, 75%, 80%, 85%, 90%, 95%, or 98% sequence identity to a native sequence set forth in Table 1. In other examples, a nucleic acid sequence involved in viral infection has a sequence that hybridizes to a sequence set forth in Table 1 and retains the activity of the sequence set forth in Table 1. For example, a nucleic acid that hybridizes to an EIF5 nucleic acid sequence set forth in Table 1 (for example the nucleic acid sequence set forth under GenBank Accession No. NM_{—}001969.3 or NM_{—}183004.3) and encodes a protein that retains EIF5 activity is contemplated by the present invention. Such sequences include the genomic sequence for the genes set forth in Table 1. The examples set forth above for EIF5 are merely illustrative and should not be limited to EIF5 as the analysis set forth in this example applies to every nucleic acid and protein listed in Table 1.

Unless otherwise specified, any reference to a nucleic acid molecule includes the reverse complement of the nucleic acid. For example, any siRNA sequence set forth herein also includes the reverse complement of that sequence. Except where single-strandedness is required by the text herein (for example, a ssRNA molecule), any nucleic acid written to depict only a single strand encompasses both strands of a corresponding double-stranded nucleic acid. For example, depiction of a plus-strand of a dsDNA also encompasses the complementary minus-strand of that dsDNA. Additionally, any reference to the nucleic acid molecule that encodes a specific protein, or a fragment thereof, encompasses both the sense strand and its reverse complement. Fragments of the nucleic acids set forth in Table 1 and throughout the specification are also contemplated. These fragments can be utilized as primers and probes to amplify, inhibit or detect any of the nucleic acids or genes set forth in Table 1.

Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that share 90% Identity)
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency (Detects Sequences that Share 80% Identity or Greater)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency (Detects Sequences that Share Greater than 50% Identity)
Hybridization: 6×SSC at RT to 55° C. for 16-20 hours
Wash at least twice: 2×-3×SSC at RT to 55° C. for 20-30 minutes each.

Also provided is a vector, comprising a nucleic acid set forth herein. The vector can direct the in vivo or in vitro synthesis of any of the proteins or polypeptides described herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene (See generally, Sambrook et al.). The vector, for example, can be a plasmid. The vectors can contain genes conferring hygromycin resistance, ampicillin resistance, gentamicin resistance, neomycin resistance or other genes or phenotypes suitable for use as selectable markers, or methotrexate resistance for gene amplification.

There are numerous other E. coli (Escherichia coli) expression vectors, known to one of ordinary skill in the art, which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. The invention provides a nucleic acid encoding a polypeptide of the present invention, wherein a yeast cell can express the nucleic acid. More specifically, the nucleic acid can be expressed by Pichia pastoris or S. cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins are known in the art and can contain genes conferring hygromycin resistance, genticin or G418 resistance, or other genes or phenotypes suitable for use as selectable markers, or methotrexate resistance for gene amplification. A number of suitable host cell lines capable of secreting intact human proteins have been developed in the art, and include the CHO cell lines, HeLa cells, COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

The expression vectors described herein can also include nucleic acids of the present invention under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as an Flp recombinase inducible promoter system, both of which are known in the art.

Insect cells also permit the expression of mammalian proteins. Recombinant proteins produced in insect cells with baculovirus vectors undergo post-translational modifications similar to that of wild-type proteins. The invention also provides for the vectors containing the contemplated nucleic acids in a host suitable for expressing the nucleic acids. The host cell can be a prokaryotic cell, including, for example, a bacterial cell. More particularly, the bacterial cell can be an E. coli cell. Alternatively, the cell can be a eukaryotic cell, including, for example, a Chinese hamster ovary (CHO) cell, a COS-7 cell, a HELA cell, an avian cell, a myeloma cell, a Pichia cell, or an insect cell. A number of other suitable host cell lines have been developed and include myeloma cell lines, fibroblast cell lines, a cell line suitable for infection by a pathogen, and a variety of tumor cell lines such as melanoma cell lines. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, Lipofectamine, or lipofectin mediated transfection, electroporation or any method now known or identified in the future can be used for other eukaryotic cellular. hosts.

### Polypeptides

The present invention provides isolated polypeptides comprising the polypeptide or protein sequences set forth under the GenBank Accession Nos. set forth in Table 1. The present invention also provides fragments of these polypeptides. These fragments can be of sufficient length to serve as antigenic peptides for the generation of antibodies. The present invention also contemplates functional fragments that possess at least one activity of a gene or gene product listed in Table 1, for example, involved in bacterial infection. It will be known to one of skill in the art that each of the proteins set forth herein possess other properties, such as for example, translation initiation activity for EIF5 or fucosyl transferase activity for FUT4, etc. Fragments and variants of the proteins set forth herein can include one or more conservative amino acid residues as compared to the amino acid sequence listed under their respective GenBank Accession Nos.

By “isolated polypeptide” or “purified polypeptide” is meant a polypeptide that is substantially free from the materials with which the polypeptide is normally associated in nature or in culture. The polypeptides of the invention can be obtained, for example, by extraction from a natural source if available (for example, a mammalian cell), by expression of a recombinant nucleic acid encoding the polypeptide (for example, in a cell or in a cell-free translation system), or by chemically synthesizing the polypeptide. In addition, a polypeptide can be obtained by cleaving full-length polypeptides. When the polypeptide is a fragment of a larger naturally occurring polypeptide, the isolated polypeptide is shorter than and excludes the full-length, naturally occurring polypeptide of which it is a fragment.

Also provided by the present invention is a polypeptide comprising an amino acid sequence at least about 50%, 55%, 60%, 65%, 70%, 75%, 80%, 85%, 90%, 95%, 96%, 97%, 98% or 99% identical to the polypeptide sequences set forth under the GenBank Accession Nos. disclosed herein.

It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b12seq/b12.html), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989, which are herein incorporated by, reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used, herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Also provided by the present invention are polypeptides set forth under the GenBank Accession Nos. disclosed herein, or fragments thereof, with one or more conservative amino acid substitutions. These conservative substitutions are such that an amino acid having similar properties replaces a naturally occurring one. Such conservative substitutions do not alter the function of the polypeptide. For example, conservative substitutions can be made as follows: Arg can be replaced with Lys, Asn can be replace with Gln, Asn can be replaced with Glu, Cys can be replaced with Ser, Gln can be replaced with Asn, Glu can be replaced with Asp, Gly can be replaced with Pro, His can be replaced with Gln, Ile can be replaced with Leu or Val, Gly can be replaced with Pro, His can be replaced with Gln, Ile can be replaced with Ile or Val, Leu can be replaced with Ile or Val, Lys can be replaced with Arg or Gln, Met can be replaced with Leu or Ile, Phe can be replaced with Met, Leu or Tyr, Ser can be replaced with Thr, Thr can be replaced with Ser, Trp can be replaced with Tyr, Tyr can be replaced with Trp or Phe; and Val can be replaced with Ile or Leu.

Thus, it is understood that, where desired, modifications and changes may be made in the nucleic acid encoding the polypeptides of this invention and/or amino acid sequence of the polypeptides of the present invention and still obtain a polypeptide having like or otherwise desirable characteristics. Such changes may occur in natural isolates or may be synthetically introduced using site-specific mutagenesis, the procedures for which, such as mis-match polymerase chain reaction (PCR), are well known in the art. For example, certain amino acids may be substituted for other amino acids in a polypeptide without appreciable loss of functional activity. It is thus contemplated that various changes may be made in the amino acid sequence of the polypeptides of the present invention (or underlying nucleic acid sequence) without appreciable loss of biological utility or activity and possibly with an increase in such utility or activity. Thus, it is clear that naturally occurring variations in the polypeptide sequences set forth herein as well as genetically engineered variations in the polypeptide sequences set forth herein are contemplated by the present invention. Cells expressing variant polypeptides, whether naturally occurring or genetically engineered can be utilized in the methods of the present invention. By providing the genomic location of genes that are involved in viral infection, the present invention has also provided the genomic location of any variant sequences of these genes. Thus, based on the information provided herein, it would be routine for one of skill in the art to identify and sequence the genomic region identified by applicants and identify variant sequences of the genes set forth herein. It would also be routine for one of skill in the art to utilize comparison tools and bioinformatics techniques to identify sequences from other species that are homologs of the genes set forth herein and are also necessary for infection, but not necessary for survival of the cell.

### Methods of Decreasing Infection

The present invention provides a method of decreasing infection in a cell by a pathogen comprising decreasing expression or activity of EIF5, HNRNPU, BST1, FBXL5, FUT4 and SNORA28.

Also provided by the present invention is a method of decreasing infection in a cell by a pathogen comprising decreasing expression or activity of one or more gene(s) or gene product(s) set forth in Table 1, i.e., EIF5, HNRNPU, BST1, FBXL5, FUT4 and SNORA28.

As stated above, an infection can be a bacterial infection, viral infection, fungal infection or a parasitic infection, to name a few. A decrease or inhibition of infection can occur in a cell, in vitro, ex vivo or in vivo. As utilized throughout, the term “infection” encompasses all phases of pathogenic life cycles including, but not limited to, attachment to cellular receptors, entry, internalization, disassembly, replication, genomic integration of pathogenic sequences, transcription of viral RNA, translation of viral RNA, transcription of host cell mRNA, translation of host cell mRNA, proteolytic cleavage of pathogenic proteins or cellular proteins, assembly of particles, endocytosis, cell lysis, budding, and egress of the pathogen from the cells. Therefore, a decrease in infection can be a decrease in attachment to cellular receptors, a decrease in entry, a decrease in internalization, a decrease in disassembly, a decrease in replication, a decrease in genomic integration of pathogenic sequences, decrease in transcription of viral RNA, a decrease in translation of viral RNA, a decrease in transcription of host cell mRNA, a decrease in translation of host cell mRNA, a decrease in proteolytic cleavage of pathogenic proteins or cellular proteins, a decrease in assembly of particles, a decrease in endocytosis, a decrease in cell lysis, a decrease in budding, or a decrease in egress of the pathogen from the cells. This decrease does not have to be complete as this can range from a slight decrease to complete ablation of the infection. A decrease in infection can be at least about 10%, 20%, 30%, 40%, 50%, 60, 70%, 80%, 90%, 95%, 100% or any other percentage decrease in between these percentages as compared to the level of infection in a control cell, for example, a cell wherein expression or activity of EIF5, HNRNPU, BST1, FBXL5, FUT4 or SNORA28 has not been decreased. A decrease in infection can be at least about 10%, 20%, 30%, 40%, 50%, 60, 70%, 80%, 90%, 95%, 100% or any other percentage decrease in between these percentages as compared to the level of infection in a control cell that has not been contacted with a compound that decreases expression or activity of EIF5, HNRNPU, BST1, FBXL5, FUT4 or SNORA28.

In the methods set forth herein, expression of EIF5, HNRNPU, BST1, FBXL5, FUT4 or SNORA28 can be inhibited, for example, by inhibiting transcription of the gene, or inhibiting translation of its gene product. Similarly, the activity of a gene product (for example, an mRNA, a polypeptide or a protein) can be inhibited, either directly or indirectly. Inhibition or a decrease in expression does not have to be complete as this can range from a slight decrease in expression to complete ablation of expression. For example, expression can be inhibited by about 10%, 20%, 30%, 40%, 50%, 60%, 70%, 80%, 90%, 95%, 99%, 100% or any percentage in between as compared to a control cell wherein the expression of EIF5, HNRNPU, BST1, FBXL5, FUT4 or SNORA28 has not been decreased or inhibited.

Similarly, inhibition or decrease in the activity of a gene product does not have to be complete as this can range from a slight decrease to complete ablation of the activity of the gene product. For example, the activity of a gene product can be inhibited by about 10%, 20%, 30%, 40%, 50%, 60%, 70%, 80%, 90%, 95%, 99%, 100% or any percentage in between as compared to a control cell wherein activity of a EIF5, HNRNPU, BST1, FBXL5, FUT4 or SNORA28 gene product has not been decreased or inhibited, or as compared to a control cell not contacted with a compound that inhibits the activity of a EIF5, HNRNPU, BST1, FBXL5, FUT4 or SNORA28 gene product. As utilized herein, “activity of a gene product” can be an activity that is involved in pathogenicity, for example, interacting directly or indirectly, with pathogen, e.g. viral protein or viral nucleic acids, or an activity that the gene product performs in a normal cell, i.e. in a non-infected cell. Depending on the gene product, one of skill in the art would know how to assay for an activity that is involved in pathogenicity, an activity that is involved in normal cellular function, or both. As set forth above, an activity of the proteins and nucleic acids listed herein can be the ability to bind or interact with other proteins. Therefore, the present invention also provides a method of decreasing infection by inhibiting or decreasing the interaction between any of the proteins of the present invention and other cellular proteins, such as, for example, transcription factors, receptors, nuclear proteins, transporters, microtubules, membrane proteins, enzymes (for example, ATPases, phosphorylases, oxidoreductases, kinases, phosphatases, synthases, lyases, aromatases, helicases, hydrolases, proteases, transferases, nucleases, ligases, reductases and polymerases) and hormones, provided that such inhibition correlates with decreasing infection by the pathogen. The present invention also provides a method of decreasing infection by inhibiting or decreasing the interaction between any of the proteins of the present invention and a cellular nucleic acid or a viral nucleic acid. Also provided is a method of decreasing infection by inhibiting or decreasing the interaction between any of the proteins of the present invention and a viral, bacterial, parasitic or fungal protein (i.e. a non-host protein).

The cells of the present invention can be prokaryotic or eukaryotic, such as a cell from an insect, fish, crustacean, mammal, bird, reptile, yeast or a bacterium, such as E. coli. The cell can be part of an organism, or part of a cell culture, such as a culture of mammalian cells or a bacterial culture. Therefore, the cell can also be part of a population of cells. The cell(s) can also be in a subject.

Examples of viral infections include but are not limited to, infections caused by RNA viruses (including negative stranded RNA viruses, positive stranded RNA viruses, double stranded RNA viruses and retroviruses) and DNA viruses. All strains, types, subtypes of DNA and RNA viruses are contemplated. herein.

Examples of RNA viruses include, but are not limited to picornaviruses, which include aphthoviruses (for example, foot and mouth disease virus O, A, C, Asia 1, SAT1, SAT2 and SAT3), cardioviruses (for example, encephalomycarditis virus and Theiller's murine encephalomyelitis virus), enteroviruses (for example polioviruses 1, 2 and 3, human enteroviruses A-D, bovine enteroviruses 1 and 2, human coxsackieviruses A1-A22 and A24, human coxsackieviruses B1-B5, human echoviruses 1-7,9,11-12, 24, 27, 29-33, human enteroviruses 68-71, porcine enteroviruses 8-10 and simian enteroviruses 1-18), erboviruses (for example, equine rhinitis virus), hepatovirus (for example human hepatitis A virus and simian hepatitis A virus), kobuviruses (for example, bovine kobuvirus and Aichi virus), parechoviruses (for example, human parechovirus 1 and human parechovirus 2), rhinovirus (for example, rhinovirus A, rhinovirus B, rhinovirus C, HRV₁₆, HRV₁₆ (VR-11757), HRV₁₄ (VR-284), or HRV_{1A} (VR-1559), human rhinovirus 1-100 and bovine rhinoviruses 1-3) and teschoviruses (for example, porcine teschovirus).

Additional examples of RNA viruses include caliciviruses, which include noroviruses (for example, Norwalk virus), sapoviruses (for example, Sapporo virus), lagoviruses (for example, rabbit hemorrhagic disease virus and European brown hare syndrome) and vesiviruses (for example vesicular exanthema of swine virus and feline calicivirus).

Other RNA viruses include astroviruses, which include mamastorviruses and avastroviruses. Togaviruses are also RNA viruses. Togaviruses include alphaviruses (for example, Chikungunya virus, Sindbis virus, Semliki Forest virus, Western equine encephalitis, Getah virus, Everglades virus, Venezuelan equine encephalitis virus and Aura virus) and rubella viruses. Additional examples of RNA viruses include the flaviviruses (for example, tick-borne encephalitis virus, Tyuleniy virus, Aroa virus, Dengue virus (types 1 to 4), Kedougou virus, Japanese encephalitis virus (JEV), West Nile virus (WNV), Kokobera virus, Ntaya virus, Spondweni virus, Yellow fever virus, Entebbe bat virus, Modoc virus, Rio Bravo virus, Cell fusing agent virus, pestivirus, GB virus A, GBV-A like viruses, GB virus C, Hepatitis G virus, hepacivirus (hepatitis C virus (HCV)) all six genotypes), bovine viral diarrhea virus (BVDV) types 1 and 2, and GB virus B).

Other examples of RNA viruses are the coronaviruses, which include, human respiratory coronaviruses such as SARS-CoV, HCoV-229E, HCoV-NL63 and HCoV-OC43. Coronaviruses also include bat SARS-like CoV, turkey coronavirus, chicken coronavirus; feline coronavirus and canine coronavirus. Additional RNA viruses include: arteriviruses (for example, equine arterivirus, porcine reproductive and respiratory syndrome virus, lactate dehyrogenase elevating virus of mice and simian hemorraghic fever virus). Other RNA viruses include the rhabdoviruses, which include lyssaviruses (for example, rabies, Lagos bat virus, Mokola virus, Duvenhage virus and European bat lyssavirus), vesiculoviruses (for example, VSV-Indiana, VSV-New Jersey, VSV-Alagoas, Piry virus, Cocal virus, Maraba virus, Isfahan virus and Chandipura virus), and ephemeroviruses (for example, bovine ephemeral fever virus, Adelaide River virus and Berrimah virus). Additional examples of RNA viruses include the filoviruses. These include the Marburg and Ebola viruses (for example, EBOV-Z, EBOV-S, EBOV-IC and EBOV-R.

The paramyxoviruses are also RNA viruses. Examples of these viruses are the rubulaviruses (for example, mumps, parainfluenza virus 5, human parainfluenza virus type 2, Mapuera virus and porcine rubulavirus), avulaviruses (for example, Newcastle disease virus), respoviruses (for example, Sendai virus, human parainfluenza virus type 1 and type 3, bovine parainfluenza virus type 3), henipaviruses (for example, Hendra virus and Nipah virus), morbilloviruses (for example, measles, Cetacean morvilliirus, Canine distemper virus, Peste-des-petits-ruminants virus, Phocine distemper virus and Rinderpest virus), pneumoviruses (for example, human respiratory syncytial virus A2, B1 and S2, bovine respiratory syncytial virus and pneumonia virus of mice), metapneumoviruses (for example, human metapneumovirus and avian metapneumovirus). Additional paramyxoviruses include Fer-de-Lance virus, Tupaia paramyxovirus, Menangle virus, Tioman virus, Beilong virus, J virus, Mossman virus, Salem virus and Nariva virus. Additional RNA viruses include the orthomyxoviruses.

These viruses include influenza viruses and strains (e.g., influenza A, influenza A strain A/Victoria/3/75, influenza A strain A/Puerto Rico/8/34, influenza A H1N1 (including but not limited to A/WS/33, A/NWS/33 and A/California/04/2009 strains) influenza B, influenza B strain Lee, and influenza C viruses) H2N2, H3N2, H5N1, H7N7, H1N2, H9N2, H7N2, H7N3 and H10N7), as well as avian influenza (for example, strains H5N1, H5N1 Duck/MN/1525/81, H5N2, H7N1, H7N7 and H9N2) thogotoviruses and isaviruses. Orthobunyaviruses (for example, Akabane virus, California encephalitis, Cache Valley virus, Snowshoe hare virus,) nairoviruses (for example, Nairobi sheep virus, Crimean-Congo hemorrhagic fever virus Group and Hughes virus), phleboviruses (for example, Candiru, Punta Toro, Rift Valley Fever, Sandfly Fever, Naples, Toscana, Sicilian and Chagres), and hantaviruses (for example, Hantaan, Dobrava, Seoul, Puumala, Sin Nombre, Bayou, Black Creek Canal, Andes and Thottapalayam) are also RNA viruses. Arenaviruses such as lymphocytic choriomeningitis virus, Lujo virus, Lassa fever virus, Argentine hemorrhagic fever virus, Bolivian hemorrhagic fever virus, Venezuelan hemorrhagic fever virus, SABV and WWAV are also RNA viruses. Boma disease virus is also an RNA virus. Hepatitis D (Delta) virus and hepatitis E are also RNA viruses.

Additional RNA viruses include reoviruses, rotaviruses, birnaviruses, chrysoviruses, cystoviruses, hypoviruses partitiviruses and totoviruses. Orbiviruses such as African horse sickness virus, Blue tongue virus, Changuinola virus, Chenuda virus, Chobar Gorge Corriparta virus, epizootic hemorraghic disease virus, equine encephalosis virus, Eubenangee virus, Teri virus, Great Island virus, Lebombo virus, Orungo virus, Palyam virus, Peruvian Horse Sickness virus, St. Croix River virus, Umatilla virus, Wad Medani virus, Wallal virus, Warrego virus and Wongorr virus are also RNA viruses.

Retroviruses include alpharetroviruses (for example, Rous sarcoma virus and avian leukemia virus), betaretroviruses (for example, mouse mammary tumor virus, Mason-Pfizer monkey virus and Jaagsiekte sheep retrovirus), gammaretroviruses (for example, murine leukemia virus and feline leukemia virus, deltraretroviruses (for example, human T cell leukemia viruses (HTLV-1, HTLV-2), bovine leukemia virus, STLV-1 and STLV-2), epsilonretriviruses (for example, Walleye dermal sarcoma virus and Walleye epidermal hyperplasia virus 1), reticuloendotheliosis virus (for example, chicken syncytial virus, lentiviruses (for example, human immunodeficiency virus (HIV) type 1, human immunodeficiency virus (HIV) type 2, human immunodeficiency virus (HIV) type 3, simian immunodeficiency virus, equine infectious anemia virus, feline immunodeficiency virus, caprine arthritis encephalitis virus and Visna maedi virus) and spumaviruses (for example, human foamy virus and feline syncytia-forming virus).

Examples of DNA viruses include polyomaviruses (for example, simian virus 40, simian agent 12, BK virus, JC virus, Merkel Cell polyoma virus, bovine polyoma virus and lymphotrophic papovavirus), papillomaviruses (for example, human papillomavirus, bovine papillomavirus, adenoviruses (for example, adenoviruses A-F, canine adenovirus type I, canined adeovirus type 2), circoviruses (for example, porcine circovirus and beak, and feather disease virus (BFDV)), parvoviruses (for example, canine parvovirus), erythroviruses (for example, adeno-associated virus types 1-8), betaparvoviruses, amdoviruses, densoviruses, iteraviruses, brevidensoviruses, pefudensoviruses, herpes viruses 1, 2, 3, 4, 5, 6, 7 and 8 (for example, herpes simplex virus 1, herpes simplex virus 2, varicella-zoster virus, Epstein-Barr virus, cytomegalovirus, Kaposi's sarcoma associated herpes virus, human herpes virus-6 variant A, human herpes virus-6 variant B and cercophithecine herpes virus 1 (B virus)), poxviruses (for example, smallpox (variola), cowpox, monkeypox, vaccinia, Uasin Gishu, camelpox, psuedocowpox, pigeonpox, horsepox, fowlpox, turkeypox and swinepox), and hepadnaviruses (for example, hepatitis B and hepatitis B-like viruses). Chimeric viruses comprising portions of more than one viral genome are also contemplated herein.

For animals, in addition to the animal viruses listed above, viruses include, but are not limited to, the animal counterpart to any above listed human virus. The provided compounds can also decrease infection by newly discovered or emerging viruses. Such viruses are continuously updated on http://en.wikipedia.org/wiki/Virus and www.virology.net.

Examples of bacterial infections include, but are not limited to infections caused by the following bacteria: Listeria (sp.), Franscicella tularensis, Mycobacterium tuberculosis, Rickettsia (all types), Ehrlichia, Chlamydia. Further examples of bacteria that can be targeted by the present methods include M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracia, Escherichia coli, Vibrio cholerae, Kingella kingae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

Examples of parasitic infections include, but are not limited to infections caused by the following parasites: Cryptosporidium, Plasmodium (all species), American trypanosomes (T. cruzi), African trypanosomes, Acanthamoeba, Entaoeba histolytica, Angiostrongylus, Anisakis, Ascaris, Babesia, Balantidium, Baylisascaris, lice, ticks, mites, fleas, Capillaria, Clonorchis, Chilomastix mesnili, Cyclspora, Diphyllobothrium, Dipylidium caninum, Fasciola, Giardia, Gnathostoma, Hetetophyes, Hymenolepsis, Isospora, Loa loa, Microsporidia, Naegleria, Toxocara, Onchocerca, Opisthorchis, Paragonimus, Baylisascaris, Strongyloides, Taenia, Trichomonas and Trichuris.

Furthermore, examples of protozoan and fungal species contemplated within the present methods include, but are not limited to, Plasmodium falciparum, other Plasmodium species, Toxoplasma gondii, Pneumocystis carinii, Trypanosoma cruzi, other trypanosomal species, Leishmania donovani, other Leishmania species, Theileria annulata, other Theileria species, Eimeria tenella, other Eimeria species, Histoplasma capsulatum, Cryptococcus neoformans, Blastomyces dermatitidis, Coccidioides immitis, Paracoccidioides brasiliensis, Penicillium marneffei, and Candida species. The provided compounds can also decrease infection by newly discovered or emerging bacteria, parasites or fungi, including multidrug resistant strains of same.

Furthermore, a decrease of expression or activity of a gene provided herein can result in a decrease in infection for two or more pathogens selected from the group consisting of the viruses, bacteria, pathogen and fungi described herein. For example, and not to be limiting, this includes two or more viruses, two ore more bacteria, two or more parasites, two or more fungi, or combinations thereof.

Further provided by the present invention is a method of inhibiting infection in a cell by a pathogen comprising decreasing expression or activity EIF5, HNRNPU, BST1, FBXL5, FUT4 or SNORA28, wherein the pathogen is a respiratory virus. Respiratory viruses include, but are not limited to, picornaviruses, orthomyxoviruses, paramyxoviruses, coronaviruses and adenoviruses. More specifically, and not to be limiting, the respiratory virus can be an influenza virus, a parainfluenza virus, an adenovirus, a rhinovirus or a respiratory syncytial virus (RSV) or any strain thereof.

Also provided by the present invention is a method of inhibiting infection in a cell by a pathogen comprising decreasing expression or activity EIF5, HNRNPU, BST1, FBXL5, FUT4 or SNORA28, wherein the pathogen is a gastrointestinal virus. Gastrointestinal viruses include, but are not limited to, picornaviruses, adenoviruses, filoviruses, flaviviruses, caliciviruses and reoviruses. More specifically, and not to be limiting, the gastrointestinal virus can be a reovirus, a Norwalk virus, an Ebola virus, a Marburg virus, an adenovirus, a rotavirus, an enterovirus, a Dengue fever virus, a yellow fever virus, or a West Nile virus.

Also provided by the present invention is a method of inhibiting infection in a cell by a pathogen comprising decreasing expression or activity of EIF5, HNRNPU, BST1, FBXL5, FUT4 or SNORA28, wherein the pathogen is a hemorraghic fever virus. These include, but are not limited to, flaviviruses, bunyaviruses, arenaviruses, filoviruses and hantaviruses. More specifically and not to be limiting, the hemorraghic fever virus can be an Ebola virus, a Marburg virus, a Dengue fever virus (types 1-4), a yellow fever virus, a Sin Nombre virus, a Junin virus, a Machupo virus, a Lassa virus, a Rift Valley fever virus, or a Kyasanur forest disease virus.

The present invention also provides a method of inhibiting infection in a cell by a pathogen comprising decreasing expression or activity of EIF5, HNRNPU, BST1, FBXL5, FUT4 or SNORA28, wherein the pathogen is a pox virus, a herpes virus, BVDV, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

Also provided is a method of inhibiting infection in a cell by a pathogen comprising decreasing expression or activity of EIF5, HNRNPU, BST1, FBXL5, FUT4 or SNORA28, wherein the pathogen is a pox virus, lymphocytic choriomeningitis virus (LCM), Junin virus, Machupo virus, Guanarito virus, Lassa Fever virus, hantavirus, Rift Valley Fever virus Ebola virus, Marburg virus or Dengue Fever virus.

The present invention also provides a method of decreasing the toxicity of a toxin in a cell comprising decreasing expression or activity of EIF5, HNRNPU, BST1, FBXL5, FUT4 or SNORA28. The cell can be in vitro; ex vivo or in vivo. Toxins can include, but are not limited to, a bacterial toxin, a neurotoxin, such as a botulinum neurotoxin, a mycotoxin, ricin, a Clostridium perfringens toxin, a Clostridium difficile toxin, a saxitoxin, a tetrodotoxin, abrin, a conotoxin, a Staphlococcal toxin, an E. coli toxin, a streptococcal toxin, a shigatoxin, a T-2 toxin, an anthrax toxin, chimeric forms of the toxins listed herein, and the like. The decrease in toxicity can be at least about 10%, 20%, 30%, 40%, 50%, 60, 70%, 80%, 90%, 95%, 100% or any other percentage decrease in between these percentages as compared to the level of toxicity in a cell wherein expression or activity of EIF5, HNRNPU, BST1, FBXL5, FUT4 or SNORA28 has not been decreased.

Toxicity can be measured, for example, via a cell viability, apopotosis assay, LDH release assay or cytotoxicity assay (See, for example, Kehl-Fie and St. Geme “Identification and characterization of an RTX toxin in the emerging pathogen Kingella kingae,” J. Bacteria 189(2):430-6 (2006) and Kirby “Anthrax Lethal Toxin Induces Human Endothelial cell Apoptosis,” Infection and Immunity 72: 430-439 (2004), both of which are incorporated herein in their entireties by this reference.)

In the methods of the present invention, expression and/or activity of EIF5, HNRNPU, BST1, FBXL5, FUT4 or SNORA28 can be decreased by contacting the cell with any composition that can decrease expression or activity. For example, the composition can be a chemical, a small or large molecule (organic or inorganic), a drug, an aptamer, a protein, a peptide, a cDNA, an antibody, a morpholino, a triple helix molecule, an siRNA, a shRNA, an miRNA, an antisense RNA, an LNA, a ribozyme or any other compound now known or identified in the future that decreases the expression and/or activity of EIF5, HNRNPU, BST1, FBXL5, FUT4 or SNORA28. A decrease in expression or activity can occur by decreasing transcription of mRNA or decreasing translation of RNA. A composition can also be a mixture or “cocktail” of two or more of the compositions described herein. A decrease in expression and/or activity can also occur by inhibiting the interaction between any of the proteins set forth in Table 1 and other cellular proteins, such as, for example, transcription factors, receptors, nuclear proteins, transporters, microtubules, membrane proteins, enzymes (for example, ATPases, phosphorylases, oxidoreductases, kinases, phosphatases, synthases, lyases, aromatases, helicases, hydrolases, proteases, transferases, nucleases, ligases, reductases and polymerases) and hormones. A decrease in expression and/or activity can also occur by inhibiting or decreasing the interaction between any of the proteins of the present invention and a cellular nucleic acid or a viral nucleic acid. A decrease can also occur by inhibiting or decreasing the interaction, either direct or indirect, between any of the proteins of the present invention and a viral, bacterial, parasitic or fungal protein (i.e. a non-host protein).

A composition can also be single composition or a mixture, cocktail or combination of two or more compositions, for example, two or more compositions selected from the group consisting of chemical, a compound, a small molecule, an inorganic molecule, an organic molecule, an aptamer, a drug, a protein, a cDNA, an antibody, a morpholino, a triple helix molecule, an siRNA, an shRNAs, an antisense nucleic acid, and LNA or a ribozyme. The two or more compositions can be the same or different types of compositions. The two or more compositions can decrease expression or activity of the same target or different targets, as one or more genes or gene products set forth in Table 1 can be modulated to effect a decrease in infection. It is understood that two or more compositions comprises three or more, four or more, five or more etc. For example, and not to be limiting two or more compositions can be an two or more compositions comprising an antisense and a small molecule; or two or more antisense molecules; or two or more small molecules; or two or more compositions comprising an siRNA and a small molecule, etc. It is understood that any combination of the types of compositions set forth herein can be utilized in the methods set forth herein.

These compositions can be used alone or in combination with other therapeutic agents such as antiviral compounds, antibacterial agents, antifungal agents, antiparasitic agents, anti-inflammatory agents, anti-cancer agents, etc. All of the compounds described herein can be contacted with a cell in vitro, ex vivo or in vivo.

Examples of antiviral compounds include, but are not limited to, amantadine, rimantadine, ribavirin, zanamavir (Relenza®) and oseltamavir (Tamiflu®) for the treatment of flu and its associated symptoms. Antiviral compounds useful in the treatment of HIV include Combivir® (lamivudine-zidovudine), maraviroc, Crixivan® (indinavir), Emtriva® (emtricitabine), Epivir® (lamivudine), Fortovase® (saquinavir-sg), Hivid® (zalcitabine), Invirase® (saquinavir-hg), Kaletra® (lopinavir-ritonavir), Lexiva™ (fosamprenavir), Norvir® (ritonavir), Retrovir® (zidovudine), Sustiva® (efavirenz), Videx EC® (didanosine), Videx® (didanosine), Viracept® (nelfinavir), Viramune® (nevirapine), Zerit® (stavudine), Ziagen® (abacavir), Fuzeon® (enfuvirtide Rescriptor® (delavirdine), Reyataz® (atazanavir), Trizivir® (abacavir-lamivudine-zidovudine), Viread® (tenofovir disoproxil fumarate), Truvada® (tenofovir-emtricitabine), Atripla® (tenofovir-emtricitabine-efavirenz) and Agenerase® (amprenavir). Other antiviral compounds useful in the treatment of Ebola and other filoviruses include ribavirin and cyanovirin-N(CV-N). For the treatment of herpes virus, Zovirax® (acyclovir) is available. Antibacterial agents include, but are not limited to, antibiotics (for example, penicillin and ampicillin), sulfa Drugs and folic acid Analogs, Beta-Lactams, aminoglycosides, tetracyclines, macrolides, lincosamides, streptogramins, fluoroquinolones, rifampin, mupirocin, cycloserine, aminocyclitol and oxazolidinones.

Antifungal agents include, but are not limited to, amphotericin, nystatin, terbinafine, itraconazole, fluconazole, ketoconazole, and griselfulvin.

Antiparasitic agents include, but are not limited to, antihelmintics, antinematodal agents, antiplatyhelmintic agents, antiprotozoal agents, amebicides, antimalarials, antitrichomonal agents, aoccidiostats and trypanocidal agents.

### Antibodies

The present invention also provides antibodies that specifically bind to the gene products, proteins and fragments thereof set forth in Table 1. The antibody of the present invention can be a polyclonal antibody or a monoclonal antibody. The antibody of the invention selectively binds a polypeptide. By “selectively binds” or “specifically binds” is meant an antibody binding reaction which is determinative of the presence of the antigen (in the present case, a polypeptide set forth in Table 1 or antigenic fragment thereof among a heterogeneous population of proteins and other biologics). Thus, under designated immunoassay conditions, the specified antibodies bind preferentially to a particular peptide and do not bind in a significant amount to other proteins in the sample. Preferably, selective binding includes binding at about or above 1.5 times assay background and the absence of significant binding is less than 1.5 times assay background.

This invention also contemplates antibodies that compete for binding to natural interactors or ligands to the proteins set forth in Table 1. In other words, the present invention provides antibodies that disrupt interactions between the proteins set forth in Table 1 and their binding partners. For example, an antibody of the present invention can compete with a protein for a binding site (e.g. a receptor) on a cell or the antibody can compete with a protein for binding to another protein or biological molecule, such as a nucleic acid that is under the transcriptional control of a transcription factor set forth in. Table 1. An antibody can also disrupt the interaction between a protein set forth in Table 1 and a pathogen, or the product of a pathogen. For example, an antibody can disrupt the interaction between a protein set forth in Table 1 and a viral protein, a bacterial protein, a parasitic protein, a fungal protein or a toxin. The antibody optionally can have either an antagonistic or agonistic function as compared to the antigen. Antibodies that antagonize pathogenic infection are utilized to decrease infection.

Preferably, the antibody binds a polypeptide in vitro, ex vivo or in vivo. Optionally, the antibody of the invention is labeled with a detectable moiety. For example, the detectable moiety can be selected from the group consisting of a fluorescent moiety, an enzyme-linked moiety, a biotin moiety and a radiolabeled moiety. The antibody can be used in techniques or procedures such as diagnostics, screening, or imaging. Anti-idiotypic antibodies and affinity matured antibodies are also considered to be part of the invention.

As used herein, the term “antibody” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody” are conjugates of antibody fragments and antigen binding proteins (single chain antibodies) as described, for example, in U.S. Pat. No. 4,704,692, the contents of which are hereby incorporated by reference.

Optionally, the antibodies are generated in other species and “humanized” for administration in humans. In one embodiment of the invention, the “humanized” antibody is a human version of the antibody produced by a germ line mutant animal. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat or rabbit having the desired specificity, affinity and capacity. In one embodiment, the present invention provides a humanized version of an antibody, comprising at least one, two, three, four, or up to all CDRs of a monoclonal antibody that specifically binds to a protein or fragment thereof set forth in Table 1. In some instances, corresponding non-human residues replace Fv framework residues of the human immunoglobulin. Humanized antibodies may also comprise residues that are found neither in the recipient antibody nor in the imported CDR or framework sequences. In general, the humanized antibody will comprise substantially all of or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. Peptides that inhibit EIF5, HNRNPU, BST1, FBXL5, FUT4 or SNORA28 expression or activity are also provided herein. Peptide libraries can be screened utilizing the screening methods set forth herein to identify peptides that inhibit activity. of any of the genes or gene products set forth in Table 1. These peptides can be derived from a protein that binds to any of the genes or gene products set forth in Table 1. These peptides can be any peptide in a purified or non-purified form, such as peptides made of D- and/or L-configuration amino acids (in, for example, the form of random peptide libraries; see Lam et al., Nature 354:82-4, 1991), phosphopeptides (such as in the form of random or partially degenerate, directed phosphopeptide libraries; see, for example, Songyang et al., Cell 72:767-78, 1993).

### Antisense Nucleic Acids

Generally, the term “antisense” refers to a nucleic acid molecule capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter).

Antisense nucleic acids are polynucleotides, for example nucleic acid molecules that are at least 6 nucleotides in length, at least 10 nucleotides, at least 15 nucleotides, at least 20 nucleotides, at least 100 nucleotides, at least 200 nucleotides, such as 6 to 100 nucleotides. However, antisense molecules can be much longer. In particular examples, the nucleotide is modified at one or more base moiety, sugar moiety, or phosphate backbone (or combinations thereof), and can include other appending groups such as peptides, or agents facilitating transport across the cell membrane (Letsinger et al., Proc. Natl. Acad. Sci. USA 1989, 86:6553-6; Lemaitre et al., Proc. Natl. Acad. Sci. USA 1987, 84:648-52; WO 88/09810) or blood-brain barrier (WO 89/10134), hybridization triggered cleavage agents (Krol et al., BioTechniques 1988, 6:958-76) or intercalating agents (Zon, Pharm. Res. 5:539-49, 1988). Additional modifications include those set forth in U.S. Pat. Nos. 7,176,296; 7,329,648; 7,262,489, 7,115,579; and 7,105,495.

Examples of modified base moieties include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine.

Examples of modified sugar moieties include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In a particular example, an antisense molecule is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-41, 1987). The oligonucleotide can be conjugated to another molecule, such as a peptide, hybridization triggered cross-linking agent, transport agent, or hybridization-triggered cleavage agent. Oligonucleotides can include a targeting moiety that enhances uptake of the molecule by host cells. The targeting moiety can be a specific binding molecule, such as an antibody or fragment thereof that recognizes a molecule present on the surface of the host cell.

In a specific example, antisense molecules that recognize a nucleic acid set forth herein, include a catalytic RNA or a ribozyme (for example see WO 90/11364; WO 95/06764; and Sarver et al., Science 247:1222-5, 1990). Conjugates of antisense with a metal complex, such as terpyridylCu (II), capable of mediating mRNA hydrolysis, are described in Bashkin et al. (Appl. Biochem Biotechnol. 54:43-56, 1995). In one example, the antisense nucleotide is a 2′-0-methylribonucleotide (Inoue et al., Nucl. Acids Res. 15:6131-48, 1987), or a chimeric RNA-DNA analogue (Inoue et al., FEBS Lett. 215:327-30, 1987).

Antisense molecules can be generated by utilizing the Antisense Design algorithm of Integrated DNA Technologies, Inc. (1710 Commercial Park, Coralville, Iowa 52241 USA; http://www.idtdna.com/Scitools/Applications/AntiSense/Antisense.aspx/)

Examples of antisense nucleic'acid molecules that can be utilized to decrease expression in the methods of the present invention, include, but are not limited to:

EIF5
(SEQ ID NO: 18)
CAGACCAGTTCTATCGCTACAAGAT
(SEQ ID NO: 19)
AAGGCAATGGAATCAAGACAGTTAT
(SEQ ID NO: 20)
CCTCCAACGTATCCCACCAAATATT
(SEQ ID NO: 21)
CAGACCCAGTTTGATGTTAAGAATG
(SEQ ID NO: 22)
GAGGAGAGGGTCAATATCCTCTTTG
(SEQ ID NO: 23)
GAGAGGGTCAATATCCTCTTTGATT
(SEQ ID NO: 24)
GGGTGTTATTGATTCATCTGACAAA
(SEQ ID NO: 25)
CATGGGCCCTCTTGTTCTAACTGAA
(SEQ ID NO: 26)
CCCTCTTGTTCTAACTGAAGTTCTT
(SEQ ID NO: 27)
CCTCTTGTTCTAACTGAAGTTCTTT
HNRNPU
(SEQ ID NO: 28)
CAAATCTCCTCAGCCACCTGTTGAA
(SEQ ID NO: 29)
GATGACACAGTGGTTTGTCTTGATA
(SEQ ID NO: 30)
ACACAGTGGTTTGTCTTGATACTTA
(SEQ ID NO: 31)
CACAGTGGTTTGTCTTGATACTTAT
(SEQ ID NO: 32)
GAGAAGATCCCAGTAAGGCATTTAT
(SEQ ID NO: 33)
CCAGAGGTAGCTGAGTGCTTTGATG
(SEQ ID NO: 34)
CATAGAGGACGTGGAGGATTCAATA
(SEQ ID NO: 35)
CCTGGGAATCGTGGCGGATATAATA
(SEQ ID NO: 36)
GGAAGTGGTGGAATCGGCTATCCAT
(SEQ ID NO: 37)
GGAGTCAGCATTATCACCAAGGATA
BST1
(SEQ ID NO: 38)
TGCTGCAGCTTCTGCTTCTACTGTT
(SEQ ID NO: 39)
GGCAGGGTTGCAGATTTCTTGAGCT
(SEQ ID NO: 40)
CAGATTTCTTGAGCTGGTGTCGACA
(SEQ ID NO: 41)
CACGAATCGAGATCTGGGTTATGCA
(SEQ ID NO: 42)
GAATCGAGATCTGGGTTATGCATGA
(SEQ ID NO: 43)
TCGAGATCTGGGTTATGCATGAAAT
(SEQ ID NO: 44)
AGCACCCATCCTGACTGTGCCTTAA
(SEQ ID NO: 45)
CACCCATCCTGACTGTGCCTTAAAG
(SEQ ID NO: 46)
CAGAACAAAGGGCGGGTCTTATCAT
(SEQ ID NO: 47)
TGCTGGCTTCCAGGACTCAACTGTA
FBXL5
(SEQ ID NO: 48)
CCGTGCTCTTCTGCAGTCTTTGTAT
(SEQ ID NO: 49)
CAGCTATCTTAATCCTCAAGAGTTA
(SEQ ID NO: 50)
CGATGCAGTCAAGTAAGCATGAAAT
(SEQ ID NO: 51)
CGGGATCGCTTTGGAAACATCTTTA
(SEQ ID NO: 52)
CATTGGGCCAGAGGTGACTGGTATA
(SEQ ID NO: 53)
TCCCGCAACTGAACTTGATACTGAA
(SEQ ID NO: 54)
CAGTATGCCTGTTTGCACGATTTAA
(SEQ ID NO: 55)
TGCTGAAGATTTGGCTGATATTGAA
(SEQ ID NO: 56)
ACGTGTACTTCTGTTTCTCAGTTTA
(SEQ ID NO: 57)
GCACCTTAATCTCTCTGGTTGTCTT
FUT4
(SEQ ID NO: 58)
GCCCGTCCCTTTCTTCCACT
(SEQ ID-NO: 59)
GTCCTTCCACTCTCCACCA
(SEQ ID NO: 60)
CCCGTCCCTTTCTTCCAC
(SEQ ID NO: 61)
TCCTTCCACTCTCCACCAAC
(SEQ ID NO: 62)
TCCTTCCACTCTCCACCAAC
(SEQ ID NO: 63)
CCTTCCACTCTCCACCAACA
(SEQ ID NO: 64)
GTCCTTCCACTCTCCACCAAC
(SEQ ID NO: 65)
GTCCTTCCACTCTCCACCAA
(SEQ ID NO: 66)
CCCGTCCCTTTCTTCCACTC
(SEQ ID NO: 67)
TCCTTCCACTCTCCACCAA
SNORA28
(SEQ ID NO: 68)
CAATTTGAGCTTGCTATAGCAAGAA
(SEQ ID NO: 69)
TGAGCTTGCTATAGCAAGAAAGTCT
(SEQ ID NO: 70)
GAGCTTGCTATAGCAAGAAAGTCTA
(SEQ ID NO: 71)
GCTATAGCAAGAAAGTCTAACCTAT
(SEQ ID NO: 72)
GCAAGAAAGTCTAACCTATTCCGGT
(SEQ ID NO: 73)
CAAGAAAGTCTAACCTATTCCGGTG
(SEQ ID NO: 74)
CCTATTCCGGTGTTCTCTCTCCCAT
(SEQ ID NO: 75)
GAGACAAGCCGTTATATAGACTTAA
(SEQ ID NO: 76)
GACAAGCCGTTATATAGACTTAAAC
(SEQ ID NO: 77)
CAAGCCGTTATATAGACTTAAACAG

Also provided are sequences comprising the antisense sequences set forth above that are not the full length mRNA for any of the genes listed in Table 1 and can be used as antisense sequences. Further provided are antisense sequences that overlap with the sequences set forth above and comprise a fragment of the above-mentioned sequences. As mentioned above, these antisense sequences are merely exemplary, as it is known to those of skill in the art that once a mRNA sequence is provided for example the mRNA sequences set forth in Table 1, it is routine to walk along the mRNA sequence to generate antisense sequences that decrease expression. Therefore, the methods of the present invention can utilize any antisense sequence that decreases the expression of a gene set forth in Table 1.

siRNAs

Short interfering RNAs (siRNAs), also known as small interfering RNAs, are double-stranded RNAs that can induce sequence-specific post-transcriptional gene silencing, thereby decreasing gene expression (See, for example, U.S. Pat. Nos. 6,506,559, 7,056,704, 7,078,196, 6,107,094, 5,898,221, 6,573,099, and European Patent No. 1,144,623, all of which are hereby incorporated in their entireties by this reference). siRNas can be of various lengths as long as they maintain their function. In some examples, siRNA molecules are about 19-23 nucleotides in length, such as at least 21 nucleotides, for example at least 23 nucleotides. In one example, siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription or translation, for example, by decreasing gene expression of EIF5, HNRNPU, BST1, FBXL5, FUT4 or SNORA28. The effects of siRNAs have been demonstrated in cells from a variety of organisms, including Drosophila, C. elegans, insects, frogs, plants, fungi, mice and humans (for example, WO 02/44321; Gitlin et al., Nature 418:430-4, 2002; Caplen et al., Proc. Natl. Acad. Sci. 98:9742-9747, 2001; and Elbashir et al., Nature 411:494-8, 2001).

Utilizing sequence analysis tools, one of skill in the art can design siRNAs to specifically target EIF5, HNRNPU, BST1, FBXL5, FUT4 or SNORA28 for decreased gene expression. siRNAs that inhibit or silence gene expression can be obtained from numerous commercial entities that synthesize siRNAs, for example, Ambion Inc. (2130 Woodward Austin, Tex. 78744-1832, USA), Qiagen Inc. (27220 Turnberry Lane, Valencia, Calif. USA) and Dharmacon Inc. (650 Crescent Drive, #100 Lafayette, Colo. 80026, USA). The siRNAs synthesized by Ambion Inc., Qiagen Inc. or Dharmacon Inc, can be readily obtained from these and other entities by providing a GenBank Accession No. for the mRNA of EIF5, HNRNPU, BST1, FBXL5, FUT4 or SNORA28. In addition, siRNAs can be generated by utilizing Invitrogen's BLOCK-IT™ RNAi Designer https://rnaidesigner.invitrogen.com/rnaiexpress.

Examples of siRNA sequences that can be utilized in the methods described herein include, but are not limited, to those set forth below. Specifically, the sense siRNA sequences set forth below and sequences complementary to these sequences can be used alone or in combination with other sequences to inhibit gene expression. Also contemplated are siRNA sequences that are shorter or longer than the sequences set forth below. For example, an siRNA sequence comprising any of the sequences set forth below can be readily generated by adding nucleotides, on one or both ends of the siRNA, that flank these sequences in the full-length mRNA for the gene of interest. Nucleotides can also be removed, from one or both ends of the siRNA to generate shorter siRNA sequences that retain their function. These sequences can comprise a 3′TT overhang and/or additional sequences that allow efficient cloning and expression of the siRNA sequences. All of the sequences disclosed herein can be cloned into vectors and utilized in vitro, ex vivo or in vivo to decrease gene expression. These siRNA sequences are merely exemplary as one of skill in the art would know that it is routine to utilize publicly available algorithms for the design of siRNA to target mRNA sequences. It is understood that any siRNA sequence set forth in the present application also includes disclosure of its reverse complement to produce siRNA duplexes. For example, the disclosure of UUCGGUUAUGCAGUCAAUUAU (SEQ ID NO: 78) also includes the disclosure of AUAAUUGACUGCAUAACCGAA (SEQ ID NO: 108); the disclosure of AGGCGCUUAAUCGGCCUCCAA (SEQ ID NO: 79) also includes the disclosure of UUGGAGGCCGAUUAAGCGCCU (SEQ ID NO: 109), etc. These sequences can then be assayed for inhibition of gene expression in vitro, ex vivo or in vivo.

EIF5
(SEQ ID NO: 78)
UUCGGUUAUGCAGUCAAUUAU
(SEQ ID NO: 79)
AGGCGCUUAAUCGGCCUCCAA
(SEQ ID NO: 80)
CAGCCAGAAGUGCAACAUGUA
(SEQ ID NO: 81)
AAGGAUGACGACAUCGAUAUU
HNRNPU
(SEQ ID NO: 82)
CUGGCCGUGGUAGUUACUCAA
(SEQ ID NO: 83)
AGGAUAUUAUUGAAUACCCAA
(SEQ ID NO: 84)
CUGGGAAUCGUGGCGGAUAUA
(SEQ ID NO: 85)
UUGGCUGGUCACUAACUACAA
BST1
(SEQ ID NO: 86)
CAGACUGCUUGUAUAUUAUCA
(SEQ ID NO: 87)
CACGGGACUGGAGGGACCAAA
(SEQ ID NO: 88)
CACGUCAUGCUGAAUGGUUCA
(SEQ ID NO: 89)
CAGCGAGAUAUCCGAGCGAGA
FBXL5
(SEQ ID NO: 90)
AUGCCUGUUUGCACGAUUUAA
(SEQ ID NO: 91)
CUGCGGAGGAAUCAAUUGCUA
(SEQ ID NO: 92)
UUGGUAAUGCCAUGUCAUUUA
(SEQ ID NO: 93)
CAGCAAGGACUAGAUUGCCUA
FUT4
(SEQ ID NO: 94)
CGGGUCCGCUACUACCACCAA
(SEQ ID NO: 95)
CCACGUGCUAUCAUUCUACUA
(SEQ ID NO: 96)
GAGGAUAGAUUAGACACUUGA
(SEQ ID NO: 97)
CACCGAGAAGCUCUGGCGCAA
SNORA28
(SEQ ID NO: 98)
ACACAAUUUGAGCUUGCUA
(SEQ ID NO: 99)
GAGCUUGCUAUAGCAAGAA
(SEQ ID NO: 100)
GCUUGCUAUAGCAAGAAAG
(SEQ ID NO: 101)
UGCUAUAGCAAGAAAGUCU
(SEQ ID NO: 102)
GCUAUAGCAAGAAAGUCUA
(SEQ ID NO: 103)
AGCAAGAAAGUCUAACCUA
(SEQ ID NO: 104)
GCAAGAAAGUCUAACCUAU
(SEQ ID NO: 105)
UCUAACCUAUUCCGGUGUU
(SEQ ID NO: 106)
ACCUAUUCCGGUGUUCUCU
(SEQ ID NO: 107)
CCAUGAGACAAGCCGUUAU

shRNA

shRNA (short hairpin RNA) is a DNA molecule that can be cloned into expression vectors to express siRNA (typically 19-29 nt RNA duplex) for RNAi interference studies. shRNA has the following structural features: a short nucleotide sequence ranging from about 19-29 nucleotides derived from the target gene, followed by a short spacer of about 4-15 nucleotides (i.e. loop) and about a 19-29 nucleotide sequence that is the reverse complement of the initial target sequence.

### Morpholinos

Morpholinos are synthetic antisense oligos that can block access of other molecules to small (about 25 base) regions of ribonucleic acid (RNA). Morpholinos are often used to determine gene function using reverse genetics methods by blocking access to mRNA. Morpholinos, usually about 25 bases in length, bind to complementary sequences of RNA by standard nucleic acid base-pairing. Morpholinos do not degrade their target RNA molecules. Instead, Morpholinos act by “steric hindrance”, binding to a target sequence within an RNA and simply interfering with molecules which might otherwise interact with the RNA. Morpholinos have been used in mammals, ranging from mice to humans.

Bound to the 5′-untranslated region of messenger RNA (mRNA), Morpholinos can interfere with progression of the ribosomal initiation complex from the 5′ cap to the start codon. This prevents translation of the coding region of the targeted transcript (called “knocking down” gene expression). Morpholinos can also interfere with pre-mRNA processing steps, usually by preventing the splice-directing snRNP complexes from binding to their targets at the borders of introns on a strand of pre-RNA. Preventing U1 (at the donor site) or U2/U5 (at the polypyrimidine moiety & acceptor site) from binding can cause modified splicing, commonly leading to exclusions of exons from the mature mRNA. Targeting some splice targets results in intron inclusions, while activation of cryptic splice sites can lead to partial inclusions or exclusions. Targets of U11/U12 snRNPs can also be blocked. Splice modification can be conveniently assayed by reverse-transcriptase polymerase chain reaction (RT-PCR) and is seen as a band shift after gel electrophoresis of RT-PCR products. Methods of designing, making and utilizing, morpholinos are disclosed in U.S. Pat. No. 6,867,349 which is incorporated herein by reference in its entirety.

### Small Molecules

Any small molecule that inhibits activity of a gene or a gene product set forth in Table 1 can be utilized in the methods of the present invention to decrease infection. These molecules are available in the scientific literature, in the StarLite/CHEMBL database available from the European Bioinformatics Institute, in DrugBank (Wishart et al. Nucleic Acids Res. 2006 Jan. 1; 34 (Database issue):D668-72), package inserts, brochures, chemical suppliers (for example, Sigma, Tocris, Aurora Fine Chemicals, to name a few), or by any other means, such that one of skill in the art makes the association between a gene product of Table 1 and inhibition of this gene product by a molecule. Preferred small molecules are those small molecules that have IC₅₀ values of less than about 1 mM, less than about 100 micromolar, less than about 75 micromolar, less than about 50 micromolar, less than about 25 micromolar, less than about 10 micromolar, less than about 5 micromolar or less than about 1 micromolar. The half maximal inhibitory concentration (IC₅₀) is a measure of the effectiveness of a compound in inhibiting biological or biochemical function. This quantitative measure indicates how much of a particular compound or other substance (inhibitor) is needed to inhibit a given biological process (or component of a process, i.e. an enzyme, cell, cell receptor or microorganism) by half. In other words, it is the half maximal (50%) inhibitory concentration (IC) of a substance (50% IC, or IC₅₀). It is commonly used as a measure of antagonist drug potency in pharmacological research. Sometimes, it is also converted to the pIC₅₀ scale (−log IC₅₀), in which higher values indicate exponentially greater potency. According to the FDA, IC₅₀ represents the concentration of a drug that is required for 50% inhibition in vitro. It is comparable to an EC₅₀ for agonist drugs. EC₅₀ also represents the plasma concentration required for obtaining 50% of a maximum effect in vivo.

The present invention also provides the synthesis of small molecules that inhibit activity of a gene product set forth in Table 1. The present invention describes gene products for which three-dimensional structures are well known and can be obtained from the RCSB Protein Databank http://www.rcsb.org/pdb/home/home.do or http://www.rcsb.org for available three-dimensional structures. Three-dimensional structures are available from the RCSB Protein Databank for example, for BST1 ([1ISF, Xray, 2.5] [1ISG, Xray, 2.6] [1ISH, Xray, 2.4] [HSI, Xray, 2.1] [1ISJ, Xray, 2.1] [1ISM, Xray, 3.0]) and EIF5 ([2E9H, NMR][2G2K, NMR][2IU1, Xray, 1.8]). For each protein set forth above, unique identifiers from the RCSB Protein Database and the resolution of the crystal structure in angstroms are provided in brackets. The structures and coordinates provided under the unique RCSB identifiers are hereby incorporated in their entireties by this reference. All of the structural information about the gene products set forth herein, for example, crystal structures and their corresponding coordinates, are readily available to one of skill in the art from the references cited herein, from the RCSB Protein Databank or elsewhere in the scientific literature.

Crystal structures can also be generated. Alternatively, one of skill in the art can obtain crystal structures for proteins, or domains of proteins, that are homologous to the proteins set forth in Table 1 from the RCSB Protein Databank or elsewhere in the scientific literature for use in homology modeling studies.

Routine high throughput in silico or in vitro screening of compound libraries for the identification of small molecules is also provided by the present invention. Compound libraries are commercially available. With an available crystal structure, it is routine for one of skill in the art to screen a library in silico and identify compounds with desirable properties, for example, binding affinity. For example, one of skill in the art can utilize the crystal structure(s) of BST1 and EIF5 in a computer program to identify compounds that bind to a site on the crystal structure with a desirable binding affinity. This can be performed in an analogous way for any protein set forth herein to identify compounds that bind with a desirable binding affinity. Numerous computer programs are available and suitable for rational drug design and the processes of computer modeling, model building, and computationally identifying, selecting and evaluating potential compounds. These include, for example, SYBYL (available from TRIPOS, St. Louis Mo.), DOCK (available from University of California, San Francisco), GRID (available form Oxford University, UK), MCSS (available from Molecular Simulations Inc., Burlington, Mass.), AUTODOCK (available from Oxford Molecular Group), FLEX X (available from TRIPOS, St. Louis Mo.), CAVEAT (available from University of California, Berkeley), HOOK (available from Molecular Simulations Inc., Burlington, Mass.), and 3-D database systems such as MACCS-3D (available from MDL Information Systems, San Leandro, Calif.), UNITY (available from TRIPOS, St. Louis Mo.), and CATALYST (available from Molecular Simulations Inc., Burlington; Mass.). Compounds can also be computationally modified using such software packages as LUDI (available from Biosym TechMA), and LEAPFROG (TRIPOS Associates, St. Louis, Mo.). These computer-modeling techniques can be performed on any suitable hardware including for example, workstations available from Silicon Graphics, Sun Microsystems, and the like. These techniques, methods, hardware and software packages are representative and are not intended to be comprehensive listing. Other modeling techniques known in the art can also be employed in accordance with this invention.

A filter can be applied to the results to yield one or more compounds with a binding affinity in a particular range, for example, and not to be limiting, from about 100 micromolar to about 100 nanomolar, from about 10 micromolar to about 10 nanomolar, from about 1 micromolar to about 1 nanomolar, or from about 0.5 micromolar to about 0.5 nanomolar. Another filter can provide compounds with a certain binding affinity and size, for example, less than 1000 daltons, less than 500 daltons, less than 400 daltons, less than 300 daltons, less than 200 daltons, less than 100 daltons or less than 50 daltons or any size in between. The ranges and properties can be modified depending on the protein being studied. The compounds identified via this screening method can be further studied in silico, in vitro or in vivo. For example, the compounds can be modified in silico and rescreened in silico to determine the effects of chemical modifications on binding affinity or other properties being assessed in silico. The compounds identified in silico can be synthesized for in vitro or in vivo analysis.

All of the screening leading up to in vivo testing can be done in silico or in combination with in vitro assays. The initial compounds identified in silico and the resulting modified compounds can be screened in vitro, for example, in cellular assays to determine the effect of the compound on the cellular host protein as well as in viral assays, to determine antiviral activity. IC₅₀ values can be obtained from the cellular assays, which may or may not be similar to the concentration necessary to effect 50% inhibition of viral infection in a viral assay. However, although not required, it is desirable to have a compound that has an IC₅₀ value of less than about 1 mM, less than about 100 micromolar, less than about 75 micromolar, less than about 50 micromolar, less than about 25 micromolar, less than about 10 micromolar, less than about 5 micromolar or less than about 1 micromolar. Similarly, although not required, it is desirable to have a compound that effects 50% inhibition of viral infection at a concentration of less than about 1 mM, less than about 100 micromolar, less than about 75 micromolar, less than about 50 micromolar, less than about 25 micromolar, less than about 10 micromolar, less than about 5 micromolar or less than about 1 micromolar or any concentration in between.

Further modifications of the compounds can be done after in vitro screening, either in silico or via chemical synthesis, for further evaluation, prior to additional in vitro screening or in vivo studies. It is understood that this process can be iterative, involving a combination of in silico and wet chemistry techniques, but routine in drug development.

Other filters can be applied to the in silico screening process, for example, a filter that takes ADMET (adsorption, distribution, metabolism, excretion, toxicity) properties into consideration can be applied. ADMET modeling can be used during compound optimization to define an acceptable property space that contains compounds likely to have the desired properties. These filters can be applied sequentially or simultaneously depending.

Libraries for virtual or in vitro screening are available for the skilled artisan, for example from ChemBridge Corporation (San Diego, Calif.), such as a GPCR library, a kinase targeted library (KINACore), or an ion channel library (Ion Channel Set), to name a few. Compound libraries can also be obtained from the National Institutes of Health. For example, the NIH Clinical Collection of compounds that have been used in clinical trials can also be screened. Biofocus DPI (Essex, United Kingdom) also maintains and designs compound libraries that can be purchased for screening. One of skill in the art can select a library based on the protein of interest. For example, a kinase library can be screened to identify a compound that binds to and/or modulates a kinase. Other libraries that target enzyme families, for example, ATPases, hydrolases, isomerases, polymerases, transferases, phosphatases, etc., can also be screened, depending on the type of enzyme.

Compound libraries can also be screened in order to identify a compound that disrupts or inhibits specific interactions, for example, the interaction of EIF5 with EIF2. See, for example, Singh et al, The EMBO Journal 2006 September; 25:4537-46. Compounds can be administered to cells comprising both EIF5 and EIF2 in order to identify compounds that disrupt this interaction and result in decreased interaction between of the two proteins. Co-immunoprecipiation experiments can be utilized. Similarly, FRET analysis can be utilized, to identify compounds that disrupt the interaction between a two proteins.

Additional inhibitors include compositions comprising carbon and hydrogen, and optionally comprising one or more of —S, —N, —O, —Br, or —Fl, appropriately bonded as a structure, with a size of less than about 1000 daltons, less than about 500 daltons, less than about 300 daltons, less than about 200 daltons, or less than about 100 daltons, that fits into a binding pocket or an active site of a gene product set forth herein. In particular, inhibitors that have the properties described in Lipinsky's Rule of Five are included herein. Lipinski's rule of five states that a drug/inhibitor has a weight under 500 Daltons, a limited lipophilicity or octanol-water partition coefficient (expressed by Log P<5, with P=[drug]org./[drug]aq.), a maximum of 5 H-bond donors (expressed as the sum of OHs and NHs), and a maximum of 10 H-bond acceptors (expressed as the sum of oxygen and nitrogen atoms). Inhibitors that violate no more than one of the above-listed five rules are also included herein.

### EIF5

The following compounds are provided as inhibitors of EIF5. More specifically, the present invention provides a method of decreasing infection by a pathogen, in a cell or a subject said method comprising administering to the cell or subject an effective amount of a compound having the structure of a formula set forth in U.S. Pat. No. 3,816,466. U.S. Pat. No. 3,816,466 is hereby incorporated in its entirety by this reference, for its disclosure of EIF5 inhibitors. An example of an EIF5 inhibitor is set forth in formula I. Compounds of formula I can be made as described in U.S. Pat. No. 3,816,466. The pathogen can be a bacterium. The pathogen can also be a virus. The virus can be a gastrointestinal virus, as described herein. The virus can be a respiratory virus as described herein.

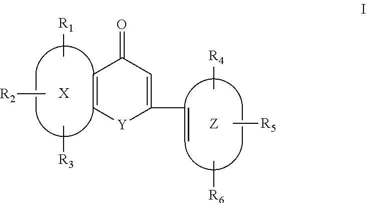

wherein Y is O or S, X and Z are aromatic or heteroaromatic rings; R1, R2, R3, R4, R5, R6 are hydrogen, halogen, amino, nitro, hydroxy, alkyl, aryl and alkoxy. More specifically, the compound can be 2-(2-amino-3-methoxyphenyl)-4-oxo-4H-[1]benzopyran, also known as 2′-amino-3′-methoxyflavone, having the structure of formula II.

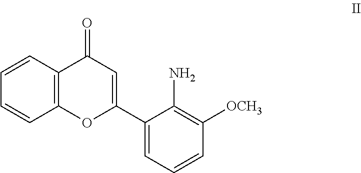 [SMILES: COc1cccc(-c2cc(=O)c3ccccc3o2)c1N]

Another EIF5 inhibitor that can be utilized is:

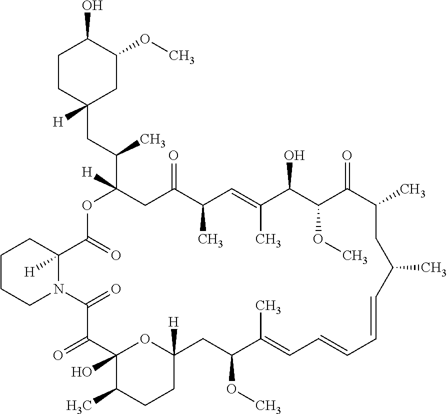

Other methods of decreasing expression and/or activity include methods of interrupting or altering transcription of mRNA molecules by site-directed mutagenesis (including mutations caused by a transposon or an insertional vector). Chemical mutagenesis can also be performed in which a cell is contacted with a chemical (for example ENU) that mutagenizes nucleic acids by introducing mutations into a gene set forth in Table 1. Transcription of mRNA molecules can also be decreased by modulating a transcription factor that regulates expression of any of the genes set forth in Table 1. Radiation can also be utilized to effect mutagenesis.

The present invention also provides decreasing expression and/or activity of a gene or a gene product set forth in Table 1 via modulation of other genes and gene products in pathways associated with the targets set forth in Table 1. Pathways include, but are not limited to ubiquitination pathways, trafficking pathways, cell signaling pathways, apoptotic pathways, TNF receptor pathways, GPCR pathways etc. Thus, other genes either upstream or downstream of the genes set forth in Table 1 are also provided herein as targets for inhibition of infection. For example, a gene product that interacts with EIF5 either upstream or downstream in the EIF5 pathway is considered a target for therapy or prevention against intracellular pathogens. For example, this can be a transcription factor that regulates expression of EIF5 or another protein that interacts, either directly (for example, via binding to EIF5) or indirectly with EIF5. Examples of genes and gene products that can be modulated in this pathway include, but are not limited to EIF2S2. These examples are merely exemplary as this applies to all of the genes and gene products set forth in Table 1 and the cellular pathways they are involved in. Table 2 provides nonlimiting examples of other genes and gene products that are associated with genes and gene products set forth in Table 1. One of skill in the art would understand that modulation, including downregulation, upregulation, inhibition or stimulation of genes and/or gene products associated with the host cellular targets set forth in Table 1 can result in inhibition of infection. Such modulation can be effected by contacting a cell with a chemical, a small or large molecule (organic or inorganic), a drug, a protein, a peptide, a cDNA, an antibody, an aptamer, a morpholino, a triple helix molecule, an siRNA, a shRNA, an miRNA, an antisense RNA, an LNA, or a ribozyme which can be obtained via the methods set forth above. A combination of a composition that decreases expression and/or activity EIF5, for example, an EIF5 inhibitor described herein, and a composition that modulates expession and/or activity of an EIF5 modulator such as EIF2S2, EIF3S4, EIF3S10, etc. is further provided. Such combinations can be utilized to effect inhibition of infection by two or more, three or more, four or more, five or more pathogens set forth herein. In particular, these combinations can be utilized to inhibit infection by two or more, three or more, four or more; or five or more viruses. More particularly, these combinations can be utilized to inhibit infection by two or more, three or more, four or more; or five or more respiratory viruses. More particularly, these combinations can be, utilized to inhibit infection by two or more, three or more, four or more; or five or more respiratory viruses selected from the group consisting of influenza, rhinovirus, adenovirus, parainfluenza virus, pox virus, RSV and measles. These combinations can also be utilized to inhibit infection by two or more strains of a respiratory virus selected from the group consisting of influenza, rhinovirus, adenovirus, parainfluenza virus, poxvirus, RSV and measles.

TABLE 2

| Gene | Interactors/Modulators |
|---|---|
| EIF5 | EIF2; EIF2S2; EIF3S4; EIF3S10; EIF2S1; EIF1AX; EIF5B; |
| | EIF3S7; EIF3S9; EIF4A1; EIF4B |
| HNRNPU | POLR2A; HNRPA0; GTF2F1; HNRPH1; HNRPF; SF3A2; |
| | SRRM1; RBMX; SFRS2; HNRPA1 |
| BST1 | NMNAT1; RYR1; CD38; NP; ENSG00000149962; NNT; |
| | NADK; ENPP1; ENPP3; NADSYN1 |
| FBXL5 | DCTN1; FBXO25; FBXO7; FBXO4; ARIH1; SUGT1; |
| | FBXO10; FBXO9; FBXW4; BRCA1 |
| FUT4 | FUT2; FUT1; ABO; SELP; FUT9; FUT6; FUT5; FUT3; |
| | B3GNT2; B4GALT1 |

### Screening Methods

The present invention provides a method of identifying a compound that binds to a gene product of EIF5, HNRNPU, BST1, FBXL5, FUT4 or SNORA28 and can decrease infection of a cell by a pathogen comprising: a) contacting a compound with a gene product of EIF5, HNRNPU, BST1, FBXL5, FUT4 or SNORA28; b) detecting binding of the compound to the gene product; and c) associating binding with a decrease in infection by the pathogen. This method can further comprise optimizing a compound that binds the gene product in an assay, for example, a cell based assay or an in vivo assay, that determines the functional ability to decrease infection. The binding assay can be a cellular assay or a non-cellular assay in which the gene product and the compound are brought into contact, for example, via immobilization of the gene product on a column, and subsequently contacting the immobilized gene product with the compound, or vice versa. Standard yeast two hybrid screens are also suitable for identifying a protein-protein interaction between a gene product set forth herein and another protein.

Further provided is a method of identifying an agent that decreases infection of a cell by a pathogen comprising: a) administering the agent to a cell containing a cellular gene encoding EIF5, HNRNPU, BST1, FBXL5, FUT4 or SNORA28; b) detecting the level and/or activity of the gene product produced by the cellular gene encoding EIF5, HNRNPU, BST1, FBXL5, FUT4 or SNORA28, a decrease or elimination of the gene product and/or gene product activity indicating an agent with antipathogenic activity. As mentioned above, a gene product activity can be binding between EIF5, HNRNPU, BST1, FBXL5, FUT4 or SNORA28 and another cellular protein or nucleic acid, or binding between EIF5, HNRNPU, BST1, FBXL5, FUT4 or SNORA28 and a pathogenic (i.e. non-host) protein.

Further provided is a method of identifying an agent that decreases infection of a cell by a pathogen comprising: a) administering the agent to a cell containing a cellular gene encoding a gene product set forth in Table 1, a decrease or elimination of the gene product and/or gene product activity indicating an agent with antipathogenic activity.

Also provided is a method of identifying an agent that decreases infection in a cell by a pathogen comprising: a) administering the agent to a cell containing a cellular gene encoding EIF5, HNRNPU, BST1, FBXL5, FUT4 or SNORA28; b) associating the agent with decreasing expression or activity of EIF5, HNRNPU, BST1, FBXL5, FUT4 or SNORA28; c) contacting the cell with a pathogen; and d) determining the level of infection, a decrease or elimination of infection indicating that the agent is an agent that decreases infection. This method can further comprise measuring the level of expression and/or activity of EIF5, HNRNPU, BST1, FBXL5, FUT4 or SNORA28.

In the methods of the present invention, if the agent has previously been identified as an agent that decreases or inhibits the level and/or activity of EIF5, HNRNPU, BST1, FBXL5, FUT4 or SNORA28, either via information in the literature, or from in vitro or in vivo results, this can indicate a decrease in infection. A decrease in infection as compared to infection in a cell that was not contacted with the agent known to decrease or inhibit the level and/or activity of the gene product can be sufficient to identify the agent as an agent that decreases or inhibits infection.

The methods described above can be utilized to identify any agent with an activity that decreases infection, prevents infection or promotes cellular survival after infection with a pathogen(s). Therefore, the cell can be contacted with a pathogen before, or after being contacted with the agent. The cell can also be contacted concurrently with the pathogen and the agent. The agents identified utilizing these methods can be used to inhibit infection in cells either in vitro, ex vivo or in vivo.

In the methods of the present invention any cell that can be infected with a pathogen can be utilized. The cell can be prokaryotic or eukaryotic, such as a cell from an insect, fish, crustacean, mammal, bird, reptile, yeast or a bacterium, such as E. coli. The cell can be part of an organism, or part of a cell culture, such as a culture of mammalian cells or a bacterial culture. The cell can also be in a nonhuman subject thus providing in vivo screening of agents that decrease infection by a pathogen. Cells susceptible to infection are well known and can be selected based on the pathogen of interest.

The test agents or compounds used in the methods described herein can be, but are not limited to, chemicals, FDA approved drugs, clinical compounds, European approved drugs, Japanese approved drugs, small molecules, inorganic molecules, organic molecules, drugs, proteins, cDNAs, large molecules, antibodies, aptamers, morpholinos, triple helix molecule, peptides, siRNAs, shRNAs, miRNAs, antisense RNAs, LNAs, ribozymes or any other compound. The compound can be random or from a library optimized to bind EIF5, HNRNPU, BST1, FBXL5, FUT4 or SNORA28. Drug libraries optimized for the proteins in the class of proteins provided herein can also be screened or tested for binding or activity. Compositions identified with the disclosed approaches can be used as lead compositions to identify other compositions having even greater antipathogenic activity. For example, chemical analogs of identified chemical entities, or variants, fragments or fusions of peptide agents, can be tested for their ability to decrease infection using the disclosed assays. Candidate agents can also be tested for safety in animals and then used for clinical trials in animals or humans.

In the methods described herein, once the cell containing a cellular gene encoding EIF5, HNRNPU, BST1, FBXL5, FUT4 or SNORA28 has been contacted with an agent, the level of infection can be assessed by measuring an antigen or other product associated with a particular infection. For example, the level of viral infection can be measured by real-time quantitative reverse transcription-polymerase chain reaction (RT-PCR) assay (See for example, Payungporn et al. “Single step multiplex real-time RT-PCR for H₅N₁ influenza A virus detection.” J Virol Methods. Sep. 22, 2005; Landolt et al. “Use of real-time reverse transcriptase polymerase chain reaction assay and cell culture methods for detection of swine influenza A viruses” Am J Vet Res. 2005 January; 66(1):119-24). If there is a decrease in infection then the composition is an effective agent that decreases infection. This decrease does not have to be complete as the decrease can be a 10%, 20%, 30%, 40%, 50%, 60%, 70%, 80%, 90%, 100% decrease or any percentage decrease in between.

In the methods set forth herein, the level of the gene product can be measured by any standard means, such as by detection with an antibody specific for the protein. The nucleic acids set forth herein and fragments thereof can be utilized as primers to amplify nucleic acid sequences, such as a gene transcript of EIF5, HNRNPU, BST1, FBXL5, FUT4 or SNORA28 by standard amplification techniques. For example, expression of a gene transcript can be quantified by real time PCR using RNA isolated from cells. A variety of PCR techniques are familiar to those skilled in the art. For a review of PCR technology, see White (1997) and the publication entitled “PCR Methods and Applications” (1991, Cold Spring Harbor Laboratory Press), which is incorporated herein by reference in its entirety for amplification methods. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods. One of skill in the art would know how to design and synthesize primers that amplify any of the nucleic acid sequences set forth herein or a fragment thereof.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified fragment, can be analyzed by one of a number of methods known in the art. The nucleic acid can be sequenced by dideoxy or other methods. Hybridization with the sequence can also be used to determine its presence, by Southern blots, dot blots, etc.

In the methods of the present invention, the level of gene product can be compared to the level of the gene product in a control cell not contacted with the compound. The level of gene product can be compared to the level of the gene product in the same cell prior to addition of the compound. The activity or the level of gene product can be compared to the activity or the level of the gene product in the same cell prior to addition of the compound. The activity or level of the gene product can also be compared to the activity or the level of the gene product in a control cell contacted with a compound known to decrease the activity and/or the level of the gene product. Activity or function, can be measured by any standard means, for example, and not to be limiting, by enzymatic assays that measure the conversion of a substrate to a product, by signal transduction assays, or binding assays that measure the binding of EIF5, HNRNPU, BST1, FBXL5, FUT4 or SNORA28 to another protein, for example.

Moreover, the regulatory region of EIF5, HNRNPU, BST1, FBXL5, FUT4 or SNORA28 can be functionally linked to a reporter gene and compounds can be screened for inhibition of reporter gene expression. Such regulatory regions can be isolated from genomic sequences and identified by any characteristics observed that are characteristic for regulatory regions of the species and by their relation to the start codon for the coding region of the gene. As used herein, a reporter gene encodes a reporter protein. A reporter protein is any protein that can be specifically detected when expressed. Reporter proteins are useful for detecting or quantitating expression from expression sequences. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), cyan fluorescent protein (CFP), red fluorescent protein (RFP) and yellow fluorescent protein (YFP).

Viral infection can also be measured via cell based assays. Briefly, by way of example, cells (20,000 to 2,500,000) are infected with the desired pathogen, and the incubation continued for 3-7 days. The antiviral agent can be applied to the cells before, during, or after infection with the pathogen. The amount of virus and agent administered can be determined by skilled practitioners. In some examples, several different doses of the potential therapeutic agent can be administered, to identify optimal dose ranges. Following transfection, assays are conducted to determine the resistance of the cells to infection by various agents. For example, if analyzing viral infection, the presence of a viral antigen can be determined by using antibody specific for the viral protein then detecting the antibody. In one example, the antibody that specifically binds to the viral protein is labeled, for example with a detectable marker such as a fluorophore. In another example, the antibody is detected by using a secondary antibody containing a label. The presence of bound antibody is then detected, for example using microscopy, flow cytometry and ELISA. In any of the methods set forth herein, the amount of viral inhibition can be compared to the amount of viral inhibition in a control cell contacted with an agent that is known to decrease viral inhibition. For example, and not to be limiting, for influenza, the amount of viral inhibition can be compared to the amount of viral inhibition in a control cell contacted with Tamiflu, amantadine, ribavirin, Relenza etc. Similar approaches can be utilized with any other virus or pathgoen for which there is a known inhibitor of viral infection that can be utilized as a positive control. Similar methods can be used to monitor bacterial, protozoal, or fungal infection (except that the antibody would recognize a bacterial, protozoal, or fungal protein, respectively).

Alternatively, or in addition, the ability of the cells to survive viral infection is determined, for example, by performing a cell viability assay, such as trypan blue exclusion or the toxicity assays described herein. Standard plaque assays can be utilized as well.

The amount of EIF5, HNRNPU, BST1, FBXL5, FUT4 or SNORA28 protein in a cell, can be determined by methods standard in the art for quantitating proteins in a cell, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

The amount of an EIF5, HNRNPU, BST1, FBXL5, FUT4 or SNORA28 nucleic acid in a cell can be determined by methods standard in the art for quantitating nucleic acid in a cell, such as in situ hybridization, quantitative PCR, RT-PCR, Taqman assay, Northern blotting, ELISPOT, dot blotting, etc., as well as any other method now known or later developed for quantitating the amount of a nucleic acid in a cell.

Any of the screening methods set forth herein can optionally comprise the step of assessing toxicity of a composition via any of the toxicity measurement methods described herein, or via any of the toxicity measurement methods known to one of skill in the art, such as, for example, the CytoTox-Glo assay (see Niles, A. et al. (2007) Anal. Biochem. 366, 197-206) or the Cell-Titer-Glo assay from Promega.

The ability of an antiviral agent to prevent or decrease infection by a virus, for example, any of the viruses listed above, can be assessed in an animal model. Several animal models for viral infection are known in the art. For example, mouse HIV models are disclosed in Sutton et al. (Res. Initiat Treat. Action, 8:22-4, 2003) and Pincus et al. (AIDS Res. Hum. Retroviruses 19:901-8, 2003); guinea pig models for Ebola infection are disclosed in Parren et al. (J. Virol. 76:6408-12, 2002) and Xu et al. (Nat. Med. 4:37-42, 1998); chimpanzee models for HCV are disclosed (Lanford et al., ILAR J. 2001; 42(2):117-26); cynomolgus monkey (Macaca fascicularis) models for influenza infection are disclosed in Kuiken et al. (Vet. Pathol. 40:304-10, 2003); mouse models for RSV are also disclosed (Sudo et al. Antivir Chem Chemother. 1999 May; 10(3):135-9) mouse models for herpes are disclosed in Wu et al. (Cell Host Microbe 22:5(1):84-94. 2009); mouse models for rhinovorus are disclosed (Bartlett et al. Nat Med. 2008 February; 14(2):199-204); pox models are disclosed in Smee et al. (Nucleosides Nucleotides Nucleic Acids 23(1-2):375-83, 2004) and in Bray et al. (J. Infect. Dis. 181(1):10-19); animal models for Dengue are available (Shresta et al. J. Virol. 2006 October; 80(20): 10208-1021; Bente and Rico-Hesse, Drug Discovery Today: Disease Models 3(1) 2006: 97-103; Martin et al. Microbiol Immunol. 2009 April; 53(4):216-23); and Franciscella tularensis models are disclosed in Klimpel et al. (Vaccine 26(52): 6874-82, 2008).

Other animal models for influenza infection are also available. These include, but are not limited to, a cotton rat model disclosed by Ottolini et al. (J. Gen. Virol., 86(Pt 10): 2823-30, 2005), as well as ferret and mouse models disclosed by Maines et al. (J. Virol. 79(18):11788-11800, 2005). One of skill in the art would know how to select an animal model for assessing the in vivo activity of an agent for its ability to decrease infection by viruses, bacteria, fungi and parasites. Such animal models can also be used to test agents for an ability to ameliorate symptoms associated with viral infection. In addition, such animal models can be used to determine the LD₅₀ and the ED₅₀ in animal subjects, and such data can be used to determine the in vivo efficacy of potential agents. LD₅₀ is an index of toxicity (lethal dose 50%), the amount of the substance that kills 50% of the test population of experimental animals when administered as a single dose. ED₅₀ is the dose of a drug that is pharmacologically effective for 50% of the population exposed to the drug or a 50% response in a biological system that is exposed to the drug. Animal models can also be used to assess antibacterial, antifungal and antiparasitic agents.

Animals of any species, including, but not limited to, insects, nematodes, eggs, birds, ferrets, cats, mice, rats, rabbits, fish (for example, zebrafish) guinea pigs, pigs, micro-pigs, goats, and non-human primates, e.g., baboons, monkeys, and chimpanzees, can be used to generate an animal model of viral infection, bacterial infection, fungal infection or parasitic infection if needed.

For example, for a model of viral infection, the appropriate animal is inoculated with the desired virus, in the presence or absence of the antiviral agent. The amount of virus and agent administered can be determined by skilled practitioners. In some examples, several different doses of the potential therapeutic agent (for example, an antiviral agent) can be administered to different test subjects, to identify optimal dose ranges. The therapeutic agent can be administered before, during, or after infection with the virus. Subsequent to the treatment, animals are observed for the development of the appropriate viral infection and symptoms associated therewith. A decrease in the development of the appropriate viral infection, or symptoms associated therewith, in the presence of the agent provides evidence that the agent is a therapeutic agent that can be used to decrease or even inhibit viral infection in a subject. For example, a virus can be tested which is lethal to the animal and survival is assessed. In other examples, the weight of the animal or viral titer in the animal can be measured. Similar models and approaches can be used for bacterial, fungal and parasitic infections.

In the methods of the present invention, the level of infection can be associated with the level of gene expression and/or activity, such that a decrease or elimination of infection associated with a decrease or elimination of gene expression and/or activity indicates that the agent is effective against the pathogen. For example, the level of infection can be measured in a cell after administration of siRNA that is known to inhibit EIF5, HNRNPU, BST1, FBXL5, FUT4 or SNORA28. If there is a decrease in infection then the siRNA is an effective agent that decreases infection. This decrease does not have to be complete as the decrease can be a 10%, 20%, 30%, 40%, 50%, 60%, 70%, 80%, 90%, 100% decrease or any percentage decrease in between. In the event that the compound is not known to decrease EIF5, HNRNPU, BST1, FBXL5, FUT4 or SNORA28 expression and/or activity, the level of expression and/or activity of EIF5, HNRNPU, BST1, FBXL5, FUT4 or SNORA28 can be measured utilizing the methods set forth above and associated with the level of infection. By correlating a decrease in EIF5, HNRNPU, BST1, FBXL5, FUT4 or SNORA28 expression with a decrease in infection, one of skill in the art can confirm that a decrease in infection is effected by a decrease in EIF5, HNRNPU, BST1, FBXL5, FUT4 or SNORA28 expression and/or activity. Similarly, the level of infection can be measured in a cell, utilizing the methods set forth above and known in the art, after administration of a chemical, small molecule, drug, protein, cDNA, antibody, shRNA, miRNA, morpholino, antisense RNA, ribozyme or any other compound. If there is a decrease in infection, then the chemical, small molecule, drug, protein, cDNA, antibody, shRNA, miRNA, morpholino, antisense RNA, LNA, ribozyme or any other compound is an effective antpathogenic agent.

The present invention provides a method of identifying an agent that can decrease infection by two or more pathogens comprising: a) administering the agent to two or more cell populations containing a cellular gene encoding a gene product set forth in Table 1; b) contacting the two or more cell populations with a pathogen, wherein each population is contacted with a different pathogen; and c) determining the level of infection, a decrease or elimination of infection by two or more pathogens indicating that the agent is an agent that decreases infection by three or more pathogens.

The present invention provides a method of identifying an agent that can decrease infection by three or more pathogens comprising: a) administering the agent to three or more cell populations containing a cellular gene encoding a gene product set forth in Table 1; b) contacting the three or more cell populations with a pathogen, wherein each population is contacted with a different pathogen; and c) determining the level of infection, a decrease or elimination of infection by three or more pathogens indicating that the agent is an agent that decreases infection by three or more pathogens.

It is understood that two or more, also means three or more, four or more, five or more, six or more, seven or more, etc. Therefore, the screening methods set forth above can be utilized to identify agents that decrease infection by four or more, five or more, six or more, seven or more pathogens set forth herein.

More particularly, the two or more, three or more; four or more, five or more, six or more, or seven or more pathogens can be selected from the group consisting of Franscicella tularensis, a picornavirus, an orthomyxovirus, a paramyxovirus, a coronavirus and an adenovirus. The two or more, three or more, four or more, five or more, six or more, or seven or more pathogens can also be selected from the group consisting of Franscicella tularensis, a filovirus, an adenovirus, a picornavirus, a calicivirus, a flavivirus and a reovirus. The two or more, three or more, four or more, five or more, six or more, or seven or more pathogens can also be selected from the group consisting of Franscicella tularensis, a picornavirus, an orthomyxovirus, a paramyxovirus, a coronavirus, an adenovirus, a filovirus, a picornavirus, a calicivirus, a flavivirus and a reovirus.

The two or more, three or more, four or more, five or more pathogens can also be selected from the group consisting of Franscicella tularensis, influenza, rhinovirus, parainfluenza virus, measles, a pox virus and RSV. The two or more, three or more, four or more, five or more, six or more, or seven or more pathogens can also be selected from the group consisting of Franscicella tularensis, a reovirus, an adenovirus, a Norwalk virus, an Ebola virus, a Marburg virus, a Dengue fever virus, a West Nile virus, a yellow fever virus, a rotavirus and an enterovirus. The two or more, three or more, four or more, five or more, six or more, or seven or more pathogens can also be selected from the group consisting of Franscicella tularensis, HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, a reovirus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus. The two or more, three or more, four or more, five or more, six or more, or seven or more pathogens can also be selected from the group consisting of Franscicella tularensis, influenza, a pox virus, LCM, Junin virus, Machupo virus, Guanarito virus, Lassa Fever virus, hantavirus, Rift Valley Fever virus Ebola virus, Marburg virus or Dengue Fever virus. The two or more, three or more, four or more, five or more, six or more, or seven or more pathogens can also be selected from the group consisting of Franscicella tularensis, an HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, tuberculosis, Yellow Fever, Rabies, Chikungunya virus or a Dengue fever virus.

The cell population used in the assay can be the same cell population for each virus strain or can be different cell populations. Typically, the agent would be administered to a different cell population for each viral strain assayed. For example, and not to be limiting, a cell population is contacted with the agent and a first virus, another cell population is contacted with the agent and second virus, a third cell population is contacted with the agent and a third virus etc. in order to determine whether the agent inhibits infection by three or more pathogens. Since the cell type will vary depending on whether or not a given virus can infect the cell, one of skill in the art would know how to pair the cell type with the virus in order to perform the assay.

This method can further comprise measuring the level of expression and/or activity of a gene product set forth in Table 1. This method can further comprise associating the level of infection with the level of expression and/or activity of a gene product set forth in Table 1. In the screening methods disclosed herein, the level of infection can be measured, for example, by measuring viral load as described in the Examples. In any of the screening methods described throughout this application, one of skill in the art can compare the level of infection in a cell contacted with a test agent with a cell contacted with a compound that is known to decrease infection in a cell, for example, a compound that targets a viral protein, in order to compare the level of infection with a positive control.

Further provided by the present invention is a method of identifying an agent that can decrease infection by three or more pathogens comprising: a) administering the agent to three or more cell populations containing a cellular gene encoding a gene product set forth in Table 1; b) contacting the three or more cell populations with a pathogen, wherein each population is contacted with a different pathogen; and c) determining the level of expression and/or activity of the gene product, a decrease or elimination of gene product expression or activity in cells indicating that the agent is an agent that decreases infection by three or more pathogens.

In the methods of the present invention, if the compound has previously been identified as a compound that decreases or inhibits the level and/or activity of the gene product, for example, via the scientific literature, in vitro studies or in vivo studies, it is not necessary to associate a decrease in infection with the level/and or activity of the gene product. A decrease in infection as compared to infection in a cell that was not contacted with the agent known to decrease or inhibit the level and/or activity of the gene product is sufficient to identify the agent as an agent that decreases or inhibits infection.

The methods described above can be utilized to identify any compound with an activity that decreases infection, prevents infection or promotes cellular survival after infection with a pathogen(s). Therefore, the cell can be contacted with a bacterium or a virus before, or after being contacted with the agent. The cell can also be contacted concurrently with the bacterium or the virus and the agent. The compounds identified utilizing these methods can be used to inhibit infection in cells either in vitro, ex vivo or in vivo.

In the methods of the present invention any cell that can be infected with a bacterium or a virus can be utilized. The cell can be prokaryotic or eukaryotic, such as a cell from an insect, fish, crustacean, mammal, bird, reptile, yeast or a bacterium, such as E. coli. The cell can be part of an organism, or part of a cell culture, such as a culture of mammalian cells or a bacterial culture. The cell can also be in a nonhuman subject thus providing in vivo screening of agents that decrease infection by a pathogen. Cells susceptible to viral infection are well known and would be selected based on the pathogen of interest.

Compositions identified with the disclosed approaches can be used as lead compositions to identify other compositions having even greater antipathogenic activity. For example, chemical analogs of identified chemical entities, or variants, fragments or fusions of peptide agents, can be tested for their ability to decrease infection using the disclosed assays. Candidate agents can also be tested for safety in animals and then used for clinical trials in animals or humans.

It is understood that any of the screening methods described herein can be performed in any tissue culture dish; including but not limited to 6 well, 12 well, 24 well, 96 well or 384 well plates. The assays can also be automated by utilizing robotics and other instrumentation standard in the art of drug screening:

### Arrays

The EIF5, HNRNPU, BST1, FBXL5, FUT4 or SNORA28 genes and nucleic acids of the invention can also be used in polynucleotide arrays. Polynucleotide arrays provide a high throughput technique that can assay a large number of polynucleotide sequences in a single sample. This technology can be used, for example, to identify samples with reduced expression of EIF5, HNRNPU, BST1, FBXL5, FUT4 or SNORA28 as compared to a control sample. This technology can also be utilized to determine the effects of reduced expression of EIF5, HNRNPU, BST1, FBXL5, FUT4 or SNORA28 on other genes. In this way, one of skill in the art can identify genes that are upregulated or downregulated upon reduction of EIF5, HNRNPU, BST1, FBXL5, FUT4 or SNORA28 expression. Similarly, one of skill in the art can identify genes that are upregulated or downregulated upon increased expression of EIF5, HNRNPU, BST1, FBXL5, FUT4 or SNORA28. This allows identification of other genes that are upregulated or downregulated upon modulation of EIF5, HNRNPU, BST1, FBXL5, FUT4 or SNORA28 expression that can be targets for therapy, such as antiviral therapy, antibacterial therapy, antiparasitic therapy or antifungal therapy.

To create arrays, single-stranded polynucleotide probes can be spotted onto a substrate in a two-dimensional matrix or array. Each single-stranded polynucleotide probe can comprise at least 6, 7, 8, 9, 10, 11, 12, 13, 14, 15, 16, 17, 18, 19, 20, 25, or 30 or more contiguous nucleotides selected from nucleotide sequences set forth under GenBank Accession Nos. herein and other nucleic acid sequences that would be selected by one of skill in the art depending on what genes, in addition to EIF5, HNRNPU, BST1, FBXL5, FUT4 or SNORA28 are being analyzed.

The array can also be a microarray that includes probes to different polymorphic alleles of EIF5, HNRNPU, BST1, FBXL5, FUT4 or SNORA28. A polymorphism exists when two or more versions of a nucleic acid sequence exist within a population of subjects. For example, a polymorphic nucleic acid can be one where the most common allele has a frequency of 99% or less. Different alleles can be identified according to differences in nucleic acid sequences, and genetic variations occurring in more than 1% of a population (which is the commonly accepted frequency for defining polymorphism) are useful polymorphisms for certain applications. The allelic frequency (the proportion of all allele nucleic acids within a population that are of a specified type) can be determined by directly counting or estimating the number and type of alleles within a population. Polymorphisms and methods of determining allelic frequencies are discussed in Hartl, D. L. and Clark, A. G., Principles of Population Genetics, Third Edition (Sinauer Associates, Inc., Sunderland Mass., 1997), particularly in chapters 1 and 2.

These microarrays can be utilized to detect polymorphic alleles in samples from subjects. Such alleles may indicate that a subject is more susceptible to infection or less susceptible to infection. For example, since the present invention shows that a disruption in EIF5, HNRNPU, BST1, FBXL5, FUT4 or SNORA28 expression results in decreased viral infection, such microarrays can be utilized to detect polymorphic versions of EIF5, HNRNPU, BST1, FBXL5, FUT4 or SNORA28 that result in decreased gene expression and/or decreased activity of the gene product to identify subjects that are less susceptible to viral infection. In addition, the existence of an allele associated with decreased expression in a healthy individual can be used to determine which genes are likely to have the least side effects if the gene product is inhibited or bound or may be selected for in commercial animals and bred into the population.

The substrate can be any substrate to which polynucleotide probes can be attached, including but not limited to glass, nitrocellulose, silicon, and nylon. Polynucleotide probes can be bound to the substrate by either covalent bonds or by non-specific interactions, such as hydrophobic interactions. Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip.™, can also be used. Use of the GeneChip.™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

### Pharmaceutical Compositions and Modes of Administration

The present invention provides a method of decreasing infection by a pathogen in a subject by decreasing the expression or activity of EIF5, HNRNPU, BST1, FBXL5, FUT4 or SNORA28 in the subject, said method comprising administering to the subject an effective amount of a composition that decreases the expression or activity of EIF5, HNRNPU, BST1, FBXL5, FUT4 or SNORA28 in the subject. It is understood that in this method, the method is not limited to the decrease in expression and/or activity of one gene or gene product, as more than one gene or gene product, for example, two, three, four, five, six etc. can be inhibited in order to inhibit infection by a pathogen.

The composition can comprise one or more of, a chemical, a compound, a small molecule, an inorganic molecule, an organice molecule, a drug, a protein, a cDNA, a peptide, an antibody, a morpholino, a triple helix molecule, an siRNA, an shRNAs, an miRNA, an antisense nucleic acid or a ribozyme that decreases the expression or activity of EIF5, HNRNPU, BST1, FBXL5, FUT4 or SNORA28.

A composition can also be a mixture, cocktail or combination of two or more compositions, for example, two or more compositions selected from the group consisting of chemical, a compound, a small molecule, an inorganic molecule, an organic molecule, an aptamer, a drug, a protein, a cDNA, an antibody, a morpholino, a triple helix molecule, an siRNA, an shRNAs, an LNA, an antisense nucleic acid or a ribozyme. The two or more compositions can be the same or different types of compositions. For example, and not to be limiting two or more compositions can be an antisense and a small molecule; or two antisense molecules; or two small molecules; or an siRNA and small molecule, etc. It is understood that any combination of the types of compositions set forth herein can be utilized in the methods set forth herein.

The present invention also provides a method of decreasing infection in a subject comprising administering to the subject an effective amount of a composition that decreases expression and/or activity of a gene product set forth in Table 1 and a composition that decreases expression and/or activity of a different gene product(s) set forth in Table 1. For example, and not to be limiting, the present invention provides a method of decreasing infection in a subject comprising administering to the subject an effective amount of a composition that decreases expression and/or activity of EIF5 and a composition that decreases expression and/or activity of HNRNPU. This is merely exemplary, as any two or more different gene product(s) set forth in Table 1 can be inhibited to decrease infection. It is understood that these methods are not limited to administration of two compositions as the combination can be a combination of two, three, four, five or six compositions, wherein, each composition comprises a chemical, a compound, a small molecule, an inorganic molecule, an organic molecule, a drug (for example, an FDA approved drug, a European Medicines Agency approved drug, a drug approved by the Japanese Pharmaceutical and Medical Device Agency) a protein, a cDNA, an aptamer, a peptide, an antibody, a morpholino, a triple helix molecule, an siRNA, an shRNAs, an miRNA, an antisense nucleic acid, an LNA or a ribozyme.

Also provided is a method of decreasing infection in a subject comprising administering to the subject an effective amount of a composition that decreases expression or activity of a gene or a gene product set forth in Table 1, wherein the composition inhibits infection by two or more pathogens. The two or more pathogens can be two or more, three or more, four or more, five or more, six or more; or seven or more bacteria, viruses, parasites, fungi or a combination thereof.

In particular, the present invention provides a method of decreasing infection in a subject comprising administering to the subject an effective amount of a composition that decreases expression or activity of a gene or a gene product set forth in Table 1, wherein the composition inhibits infection by two or more, three or more, four or more, five or more, six or more; or seven or more viruses. Table 3 shows gene products set forth in Table 1 for which at least 50% inhibition of viral replication was observed via siRNA experiments or gene trap experiments as indicated by an X. It is understood that one of skill in the art can select a gene product from Table 3 and decrease its expression or activity to reduce infection by one or more viruses marked with an X, for that gene product. One of skill in the art can readily select the gene product(s) in Table 3 that can be inhibited in order to effect inhibition of infection by two or more, three or more, four or more, five or more, six or more, seven or more viruses etc.

The pathogens are not restricted to the bacteria and viruses set forth in Table 3 as this data can be combined with data for a composition that decreases expression and/or activity of a gene product from Table 1 and inhibits any other bacteria, virus, fungi or parasite not listed in Table 3. For example, data for EIF5 can be combined with data showing that decreased expression and/or activity of EIF5 leads to inhibition of HCV infection, thus showing that activity or expression of EIF5 can be decreased to inhibit infection by tularemia, influenza, cowpox, rhinovirus, Dengue, and HCV. This example is merely exemplary as data for any gene product set forth in Table 3 can be combined with data for any other virus, bacteria, fungi or parasite.

Therefore, the present invention provides a method of decreasing infection in a subject comprising administering to the subject an effective amount of a composition that decreases expression or activity of a gene or a gene product set forth in Table 1, wherein the composition inhibits infection by two or more, three or more, four or more, five or more, six or more; or seven or more pathogens, wherein at least two or more of the pathogens are selected from the group consisting of tularemia, influenza, RSV, HSV, cowpox, rhinovirus, Dengue and HIV. Also provided is a method of decreasing infection in a subject comprising administering to the subject an effective amount of a composition that decreases expression or activity of a gene or a gene product set forth in Table 1, wherein the composition inhibits infection by two or more, three or more, four or more, five or more, six or more; or seven or more pathogens, wherein at least two or more of the pathogens are selected from the group consisting of tularemia, influenza, RSV, HSV, cowpox, rhinovirus, Dengue, HIV, and HCV.

TABLE 3

| Gene ID | Gene | Tularemia | Flu | RSV | HSV2 | Cowpox | R-16 | Dengue | HIV | Spectrum |
|---|---|---|---|---|---|---|---|---|---|---|
| 1983 | EIF5 | X | X | | | X | X | X | | 5 |
| 3192 | HNRNPU | X | X | | X | X | X | X | X | 7 |
| 683 | BST1 | X | | | X | X | | X | | 4 |
| 26234 | FBXL5 | X | | X | X | | X | X | | 5 |
| 2526 | FUT4 | X | | | | X | X | X | | 4 |
| 677811 | SNORA28 | X | | | | X | X | | | 3 |

Combinations of gene products can be inhibited in a cell or in a subject to achieve inhibition of two or more, three or more, four or more, five or more, six or more, seven or more viruses etc. Any combination of compositions that decrease expression and/or activity of two or more, three or more, four or more, five or more, six or more gene products set forth in Table 1 or 3 can be administered to inhibit infection by two or more, three or more, four or more, five or more or six or more viruses. For example, and not to be limiting, a composition that decreases expression and/or activity of HNRNPU can be administered to inhibit infection by any two or More viruses selected from the group consisting of tularemia, influenza, HSV, pox, rhinovirus, Dengue, and HIV. In another example, a composition that decreases expression and/or activity of EIF5 can be administered in combination with a composition that decreases expression and/or activity of FBXL5 to inhibit infection by any two or more viruses selected from the group consisting of tularemia, influenza, RSV, HSV, pox, rhinovirus, Dengue, and HIV.

Also provided is a method of decreasing infection in a subject comprising administering to the subject an effective amount of a composition that decreases expression or activity of a gene or a gene product set forth in Table 1, wherein the composition inhibits infection by two or more respiratory viruses. Also provided is a method of decreasing infection in a subject comprising administering to the subject an effective amount of a composition that decreases expression or activity of a gene or a gene product set forth in Table 1, wherein the composition inhibits infection by three or more respiratory viruses. Also provided is a method of decreasing infection in a subject comprising administering to the subject an effective amount of a composition that decreases expression or activity of a gene or a gene product set forth in Table 1, wherein the composition inhibits infection by four or more respiratory viruses. Also provided is a method of decreasing infection in a subject comprising administering to the subject an effective amount of a composition that decreases expression or activity of a gene or a gene product set forth in Table 1, wherein the composition inhibits infection by five or more respiratory viruses. These can be selected from the group consisting of: a picornavirus, an orthomyxovirus, a paramyxovirus, a coronavirus and an adenovirus. Since picornaviruses, orthomyxoviruses, paramyxoviruses, coronaviruses and adenoviruses are families of viruses, two or more, three or more, four or more, or five or more respiratory viruses can be from the same or from different families. For example, and not to be limiting, the composition can inhibit infection by two or more orthomyxoviruses; two or more picornaviruses; an orthomyxovirus, an adenovirus, and a picornavirus; an orthomyxovirus, a paramyxovirus and an adenovirus; an orthomyxovirus, two picornaviruses and a paramyxovirus; three orthomyxoviruses, a picornavirus and an adenovirus, etc. More particularly, the composition can inhibit infection by two or more, three or more or four or more respiratory viruses selected from the group consisting of an influenza virus, a measles virus, a pox virus, a parainfluenza virus, an adenovirus, a rhinovirus and an RSV virus. The composition can also inhibit infection by two or more strains of a respiratory virus selected from the group consisting, of influenza virus, a measles virus, a pox virus, a parainfluenza virus, an adenovirus, a rhinovirus and an RSV virus. For example, the composition can inhibit infection by two or more, three or more, four or more, five or more; or six or more strains of influenza selected from the group consisting of H1N1, H2N2, H3N2, H5N1, H7N7, H1N2, H9N2, H7N2, H7N3, H10N7, H5N1, H5N2, H7N1, H7N7 and H9N2.

Further provided is a method of decreasing infection in a subject comprising administering to the subject an effective amount of a composition that decreases expression or activity of EIF5 or HNRNPU, wherein the composition inhibits infection by influenza and rhinovirus.

Further provided is a method of decreasing infection in a subject comprising administering to the subject an effective amount of a composition that decreases expression or activity of FBXL5, wherein the composition inhibits infection by RSV and rhinovirus.

The present invention also provides a method of decreasing infection in a subject comprising administering to the subject an effective amount of a composition that decreases expression or activity of a gene or a gene product set forth in Table 1, wherein the composition inhibits infection by two or more gastrointestinal viruses. The present invention also provides a method of decreasing infection in a subject comprising administering to the subject an effective amount of a composition that decreases expression or activity of a gene or a gene product set forth in Table 1, wherein the composition inhibits infection by three or more gastrointestinal viruses. The present invention also provides a method of decreasing infection in a subject comprising administering to the subject an effective amount of a composition that decreases expression or activity of a gene or a gene product set forth in Table 1, wherein the composition inhibits infection by four or more gastrointestinal viruses. The present invention also provides a method of decreasing infection in a subject comprising administering to the subject an effective amount of a composition that decreases expression or activity of a gene or a gene product set forth in Table 1, wherein the composition inhibits infection by five or more gastrointestinal viruses. These viruses can be selected from the group consisting of: a filovirus, an adenovirus, a picornavirus, a calicivirus, a flavivirus or a reovirus. Since filoviruses, adenoviruses, picornaviruses, caliciviruses, flaviviruses and reoviruses are families of viruses, the composition can inhibit infection by two or more, three or more, four or more, or five or more gastrointestinal viruses from the same or from different families. More particularly, the composition can inhibit infection by two or more, three or more, four or more, or five or more gastrointestinal viruses selected from the group consisting of a reovirus, an adenovirus, a Norwalk virus, an Ebola virus, a Marburg virus, a Dengue fever virus, a West Nile virus, a yellow fever virus, a rotavirus and an enterovirus.

The present invention also provides a method of decreasing infection in a subject comprising administering to the subject an effective amount of a composition that decreases expression or activity of a gene or a gene product set forth in Table 1, wherein the composition inhibits infection by one or more pathogens selected from the group consisting of: a picornavirus, an orthomyxovirus, a paramyxovirus, a coronavirus, an adenovirus, and inhibits infection by one or more pathogens selected from the group consisting of: a flavivirus, an adenovirus, a filovirus, a calicivirus or a reovirus.

The present invention also provides a method of decreasing infection in a subject comprising administering to the subject an effective amount of a composition that decreases expression or activity of a gene or a gene product set forth in Table 1, wherein the composition inhibits infection by two or more, three or more, four or more, five or more; or six or more pathogens selected from the group consisting of Franciscella tularensis, HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by two or more, three or more, four or more, five or more; or six or more pathogens selected from the group consisting of: Franciscella tularensis, influenza, a pox virus, LCM, Junin virus, Machupo virus, Guanarito virus, Lassa Fever virus, hantavirus, Rift Valley Fever virus Ebola virus, Marburg virus or Dengue Fever virus.

The present invention also provides a method of decreasing infection in a subject comprising administering to the subject an effective amount of a composition that decreases expression or activity of a gene or a gene product set forth in Table 1, wherein the composition inhibits infection by three or more pathogens. The three or more pathogens can be selected from the viruses, bacteria, parasites and fungi set forth herein. More particularly, the three or more pathogens can be selected from the group consisting of: Franciscella tularensis, an HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by four or more pathogens. The four or more pathogens can be selected from the viruses, bacteria, parasites and fungi set forth herein. More particularly, the four or more pathogens can be selected from the group consisting of: Franciscella tularensis, an HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by five or more pathogens. The five or more pathogens can be selected from the viruses, bacteria, parasites and fungi set forth herein. More particularly, the five or more pathogens can be selected from the group consisting of: Franciscella tularensis; an HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits infection by six or more pathogens. The six or more pathogens can be selected from the viruses, bacteria, parasites and fungi set forth herein. More particularly, the six or more pathogens can be selected from the group consisting of: Franciscella tularensis, an HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The present invention also provides a method of decreasing infection in a subject comprising administering to the subject an effective amount of a composition that decreases expression or activity of a gene or a gene product set forth in Table 1, wherein the composition inhibits co-infection by HIV and one or more viruses, bacteria, parasites or fungi. For example, decreasing co-infection of HIV and Franciscella tularensis. For further example, decreasing co-infection of HIV and any of the viruses, including for example any families, genus, species, or group of viruses is also provided. As a further example, co-infection of HIV and a respiratory virus is provided herein. Respiratory viruses include picornaviruses, orthomyxoviruses, paramyxoviruses, coronaviruses, and adenoviruses. More specifically, the respiratory virus can be any strain of influenza, rhinovirus, adenovirus, a pox virus, measles, parainfluenza virus or RSV. In particular, use of a composition that decreases expression or activity of a gene or a gene product set forth in Table 1, wherein the composition inhibits co-infection of HIV and any strain of influenza is provided herein. Also provided is decreasing co-infection of HIV and a gastrointestinal virus. Gastrointestinal viruses include picornaviruses, filoviruses, flaviviruses, caliciviruses and reoviruses. More specifically, and not to be limiting, the gastrointestinal virus can be any strain of reovirus, a Norwalk virus, an Ebola virus, a Marburg virus, a rotavirus, an enterovirus, a Dengue fever virus, a yellow fever virus, or a West Nile virus. Further provided is a method of decreasing co-infection of HIV with a pox virus, LCM, Junin virus, Machupo virus, Guanarito virus, Lassa Fever virus, hantavirus, Rift Valley Fever virus Ebola virus, Marburg virus or Dengue Fever virus. More particularly, decreasing co-infection of HIV and a hepatitis virus, such as Hepatitis A, Hepatitis B or Hepatitis C is provided. Also provided is decreasing co-infection of HIV and a herpes virus, for example, HSV-1 or HSV-2. In addition decreasing co-infection of HIV and tuberculosis is also provided. Further provided is decreasing co-infection of HIV and CMV, as well as decreasing co-infection of HIV and HPV.

As described herein, the genes set forth in Table 1 can be involved in the pathogenesis of two or more respiratory viruses. Therefore, the present invention provides methods of treating or preventing an unspecified respiratory infection in a subject by administering a composition that decreases activity or expression of a gene involved in the pathogenesis of two or more respiratory viruses. More particularly, the present invention provides a method of decreasing an unspecified respiratory infection in a subject comprising: a) diagnosing a subject with an unspecified respiratory infection; and b) administering to the subject an effective amount of a composition that decreases expression or activity of a gene or a gene product set forth in Table 1, wherein the composition inhibits infection by two or more respiratory viruses selected from the group consisting of picornaviruses, orthomyxoviruses, paramyxoviruses, coronaviruses, or adenoviruses.

In particular, the gene or gene product in this method can be selected from the group consisting of EIF5, HNRNPU, BST1, FBXL5, FUT4, and SNORA28.

As set forth above, in the methods of the present invention, the two or more respiratory viruses can be from the same family or from a different family of respiratory viruses. More specifically, the respiratory virus can be any strain of influenza, rhinovirus, adenovirus, a pox virus, parainfluenza virus or RSV. In this method, the composition can be a composition that inhibits infection by three or more, four or more, five or more; or six or more respiratory viruses selected from the group consisting of picornaviruses; orthomyxoviruses, paramyxoviruses, coronaviruses, or adenoviruses.

As described herein, the genes set forth in Table 1 can be involved in the pathogenesis of two or more gastrointestinal viruses. Therefore, the present invention provides methods of treating or preventing an unspecified gastrointestinal infection in a subject by administering a composition that decreases activity or expression of a gene involved in the pathogenesis of two or more gastrointestinal viruses. More particularly, the present invention provides a method of decreasing an unspecified gastrointestinal infection in a subject comprising: a) diagnosing a subject with an unspecified gastrointestinal infection; and b) administering to the subject an effective amount of a composition that decreases expression or activity of a gene or a gene product set forth in Table 1, wherein the composition inhibits infection by two or more gastrointestinal viruses selected from the group consisting of a flavivirus, a filovirus, a calicivirus or a reovirus. As set forth above, in the methods of the present invention, the two or more gastrointestinal viruses can be from the same family or from a different family of gastrointestinal viruses. More particularly, and not to be limiting, the gastrointestinal virus can be any strain of reovirus, a Norwalk virus, an Ebola virus, a Marburg virus, a rotavirus, an enterovirus, a Dengue fever virus, a yellow fever virus, or a West Nile virus. In this method, the composition can be a composition that inhibits infection by three or more, four or more, five or more; or six or more gastrointestinal viruses selected from the group consisting of a flavivirus, a filovirus, a calicivirus or a reovirus.

The present invention also provides a method of preventing or decreasing an unspecified pandemic or bioterrorist threat in a subject comprising: a) diagnosing a subject with an unspecified pandemic or bioterrorist inflicted infection; and b) administering to the subject an effective amount of a composition that decreases expression or activity of a gene or a gene product set forth in Table 1, wherein the composition inhibits infection by two or more, three or more, four or more; or five or more viruses selected from the group consisting of a pox virus, an influenza virus, West Nile virus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus and a Dengue fever virus.

Also provided by the present invention is a method of managing secondary infections in a patient comprising administering to the subject an effective amount of a composition that decreases expression or activity of a gene or a gene product set forth in Table 1, wherein the composition can inhibit infection by HIV and one or more, two or more, three or more, four or more; or five or more secondary infections.

As set forth above, the genes set forth in Table 1 can be involved in the pathogenesis of three or more pathogens. Therefore, the present invention provides methods of treating or preventing an unspecified infection by administering a composition that decreases the activity or expression of a gene that is involved in the pathogenesis of three or more pathogens. Therefore, the present invention provides a method of decreasing infection in a subject comprising: a) diagnosing a subject with an unspecified infection and; b) administering a composition that decreases the expression or activity of a gene or gene product set forth in Table 1, wherein the composition decreases infection by three or more pathogens. More specifically, the three or more pathogens can be selected from the group consisting of: Francicella tularensis, an HIV, a pox virus, a herpes virus, a reovirus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, Yellow Fever, Rabies, Chikungunya virus or a Dengue fever virus.

The infection can be a viral infection, a parasitic infection, a bacterial infection or a fungal infection, to name a few. As utilized herein, “an unspecified infection” is an infection that presents symptoms associated with an infection, but is not identified as specific infection. One of skill in the art, for example, a physician, a nurse, a physician's assistant, a medic or any other health practitioner would know how to diagnose the symptoms of infection even though the actual pathogen may not be known. For example, the patient can present with one or more symptoms, including, but not limited to, a fever, fatigue, lesions, weight loss, inflammation, a rash, pain (for example, muscle ache, headache, ear ache, joint pain, etc.), urinary difficulties, respiratory symptoms (for example, coughing, bronchitis, lung failure, breathing difficulties, bronchiolitis, airway obstruction, wheezing, runny nose, sinusitis, congestion, etc.), gastrointestinal symptoms for example, nausea; diarrhea; vomiting, dehydration, abdominal pain, intestinal cramps, rectal bleeding, etc.), This can occur in the event of a bioterrorist attack or a pandemic. In this event, one of skill in the art would know to administer a composition that inhibits infection by decreasing the expression or activity of a gene or gene product set forth in Table 1 that is involved in the pathogenesis of several pathogens. Similarly, if there is a threat of an unspecified infection, for example, a threat of a bioterrorist attack, a composition that decreases the expression or activity of a gene or gene product set forth in Table 1 can be administered prophylactically to a subject to prevent an unspecified infection in a subject.

Throughout this application, by “treat,” “treating,” or “treatment” is meant a method of reducing the effects of an existing infection. Treatment can also refer to a method of reducing the disease or condition itself rather than just the symptoms. The treatment can be any reduction from native levels and can be, but is not limited to, the complete ablation of the disease or the symptoms of the disease. Treatment can range from a positive change in a symptom or symptoms of viral infection to complete amelioration of the viral infection as detected by art-known techniques. For example, a disclosed method is considered to be a treatment if there is about a 10% reduction in one or more symptoms of the disease in a subject with the disease when compared to native levels in the same subject or control subjects. Thus, the reduction can be about a 10, 20, 30, 40, 50, 60, 70, 80, 90, 100%, or any amount of reduction in between as compared to native or control levels.

The methods of the present invention can also result in a decrease in the amount of time that it normally takes to see improvement in a subject. For example, a decrease in infection can be a decrease of hours, a day, two days, three days, four days, five days, six days, seven days, eight days, nine days, ten days, eleven days, twelve days, thirteen days, fourteen days, fifteen days or any time in between that it takes to see improvement in the symptoms, viral load or any other parameter utilized to measure improvement in a subject. For example, if it normally takes 7 days to see improvement in a subject not taking the composition, and after administration of the composition, improvement is seen at 6 days, the composition is effective in decreasing infection. This example is not meant to be limiting as one of skill in the art would know that the time for improvement will vary depending on the infection.

As utilized herein, by “prevent,” “preventing,” or “prevention” is meant a method of precluding, delaying, averting, obviating, forestalling, stopping, or hindering the onset, incidence, severity, or recurrence of infection. For example, the disclosed method is considered to be a prevention if there is about a 10%, reduction in onset, incidence, severity, or recurrence of infection, or symptoms of infection (e.g., inflammation, fever, lesions, weight loss, etc.) in a subject exposed to an infection when compared to control subjects exposed to an infection that did not receive a composition for decreasing infection. Thus, the reduction in onset, incidence, severity, or recurrence of infection can be about a 10, 20, 30, 40, 50, 60, 70, 80, 90, 100%, or any amount of reduction in between as compared to control subjects. For example, and not to be limiting, if about 10% of the subjects in a population do not become infected as compared to subjects that did not receive preventive treatment, this is considered prevention.

Also provided is a method of decreasing infection in a subject comprising: a) administering a composition that decreases the expression or activity of a gene or gene product set forth in Table 1, in a subject with an unspecified infection; b) diagnosing the type of infection in the subject and; c) administering a composition that decreases the expression or activity of a gene or a gene product set forth in Table 1 for the diagnosed infection.

Further provided is a method of treating viral infection comprising: a) diagnosing a subject with a viral infection; and b) removing a drug from the subject that decreases the expression or activity of a gene or gene product set forth in Table 1, if the viral infection is not a viral infection that is inhibited by a composition that decreases the expression or activity of a gene or gene product set forth in Table 1. As mentioned above, upon recognizing that a subject has an infection or the symptoms of an infection, for example, in the case of a bioterrorist attack or a pandemic, given that a gene or gene product set forth in Table 1 can be involved in the pathogenesis of several pathogens, a practitioner can prescribe or administer a composition that decreases the expression or activity of the gene or gene product. After administration, the practitioner, who can be the same practitioner or a different practitioner, can diagnose the type of infection in a subject. This diagnosis can be a differential diagnosis where the practitioner distinguishes between infections by comparing signs or symptoms and eliminates certain types of infection before arriving at the diagnosis for a specific infection, or a diagnosis based on a test that is specific for a particular infection. Once a specific infection is diagnosed, if the gene or gene product is involved in the pathogenesis of this infection, the practitioner can prescribe or administer a composition that decreases the expression or activity of that gene or gene product. This can be the same composition administered prior to diagnosis of the specific infection or a different composition that decreases expression or activity.

Also provided is a method of preventing infection in a subject comprising administering to a subject susceptible to an unspecified infection a composition that decreases the expression or activity of a gene or gene product set forth in Table 1. The composition can be administered in response to a lethal outbreak of an infection. For example, the infection can be a pandemic or a bioterrorist created infection. If there is a threat of an unspecified infection, such as a viral infection, a bacterial infection, a parasitic infection or an infection by a chimeric pathogenic agent, to name a few, a composition can be administered prophylactically to a subject to prevent an unspecified infection in a subject. The threat can also come in the form of a toxin. One of skill in the art would know to administer a composition that inhibits infection by decreasing the expression or activity of any gene or gene product set forth in Table 1 that is involved in the pathogenesis of two or more, three or more, four or more; or five or more pathogens.

Such prophylactic use can decrease the number of people in a population that are infected, thus preventing further spread of a pandemic or decreasing the effects of a bioterrorist attack.

The composition(s) can be administered before or after infection. The decrease in infection in a subject need not be complete as this decrease can be a 10%, 20%, 30%, 40%, 50%, 60%, 70%, 80%, 90%, 100% or any other percentage decrease in between as long as a decrease occurs. This decrease can be correlated with amelioration of symptoms associated with infection. These compositions can be administered to a subject alone or in combination with other therapeutic agents described herein, such as anti-viral compounds, antibacterial agents, antifungal agents, antiparasitic agents, anti-inflammatory agents, anti-cancer agents, etc. Examples of viral infections, bacterial infections, fungal infections parasitic infections are set forth above. The compounds set forth herein or identified by the screening methods set forth herein can be administered to a subject to decrease infection by any pathogen or infectious agent set forth herein. Any of the compounds set forth herein or identified by the screening methods of the present invention can also be administered to a subject to decrease infection by any pathogen, now known or later discovered in which EIF5, HNRNPU, BST1, FBXL5, FUT4 or SNORA28 is involved.

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Methods of introduction include, but are not limited to, mucosal, topical, intradermal, intrathecal, intratracheal, via nebulizer, via inhalation, intramuscular, intraperitoneal, vaginal, rectal, intravenous, subcutaneous, intranasal, and oral routes. The compounds can be administered by any convenient route, for example by infusion or bolus injection, by absorption through epithelial or mucocutaneous linings (for example, oral mucosa, rectal, vaginal and intestinal mucosa, etc.) and can be administered together with other biologically active agents. Administration can be systemic or local. Pharmaceutical compositions can be delivered locally to the area in need of treatment, for example by topical application or local injection.

Pharmaceutical compositions are disclosed that include a therapeutically effective amount of an RNA, DNA, antisense molecule, an LNA, ribozyme, siRNA, shRNA molecule, miRNA molecule, drug, an aptamer, protein, small molecule, peptide inorganic molecule, organic molecule, antibody or other therapeutic agent, alone or with a pharmaceutically acceptable carrier. Furthermore, the pharmaceutical compositions or methods of treatment can be administered in combination with (such as before, during, or following) other therapeutic treatments, such as other antiviral agents, antibacterial agents, antifungal agents and antiparasitic agents.

For all of the administration methods disclosed herein, each method can optionally comprise the step of diagnosing a subject with an infection or diagnosing a subject in need of prophylaxis or prevention of infection.

### Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the therapeutic agents herein disclosed. In general, the nature of the carrier will depend on the mode of administration being employed. For instance, parenteral formulations usually include injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (for example powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

Embodiments of the disclosure including medicaments can be prepared with conventional pharmaceutically acceptable carriers, adjuvants and counterions as would be known to those of skill in the art.

The amount of therapeutic agent effective in decreasing or inhibiting infection can depend on the nature of the pathogen and its associated disorder or condition, and can be determined by standard clinical techniques. Therefore, these amounts will vary depending on the type of virus, bacteria, fungus, parasite or other pathogen. In addition, in vitro assays can be employed to identify optimal dosage ranges. The precise dose to be employed in the formulation will also depend on the route of administration, and the seriousness of the disease or disorder, and should be decided according to the judgment of the practitioner and each subject's circumstances. For example, dosage can range from 0.01 mg/kg to 100 mg/kg/. Multiple administrations and/or dosages can also be used. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In an example in which a nucleic acid is employed to reduce infection, such as an antisense or siRNA molecule, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). siRNA carriers also include, polyethylene glycol (PEG), PEG-liposomes, branched carriers composed of histidine and lysine (HK polymers), chitosan-thiamine pyrophosphate carriers, surfactants (for example, Survanta and Infasurf), nanochitosan carriers, and D5W solution. The present disclosure includes all forms of nucleic acid delivery, including synthetic oligos, naked DNA, plasmid and viral delivery, integrated into the genome or not.

As mentioned above, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells a nucleic acid, for example an antisense molecule or siRNA. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), and pseudotyped retroviral vectors (Agrawal et al., Exper. Hematol. 24:738-747, 1996). Other nonpathogenic vector systems such as the foamy virus vector can also be utilized (Park et al. “Inhibition of simian immunodeficiency virus by foamy virus vectors expressing siRNAs.” Virology. 2005 Sep. 20). It is also possible to deliver short hairpin RNAs (shRNAs) via vector delivery systems in order to inhibit gene expression (See Pichler et al. “In vivo RNA interference-mediated ablation of MDR1 P-glycoprotein.” Clin Cancer Res. 2005 Jun. 15; 11 (12):4487-94; Lee et al. “Specific inhibition of HIV-1 replication by short hairpin RNAs targeting human cyclin T1 without, inducing apoptosis.” FEBS Lett. 2005 Jun. 6; 579(14):3100-6.).

Physical transduction techniques can also be used, such as liposome delivery and receptor-mediated and other endocytosis mechanisms (see, for example, Schwartzenberger et al., Blood 87:472-478, 1996) to name a few examples. This invention can be used in conjunction with any of these or other commonly used gene transfer methods.

### Transgenic Cells and Non-Human Mammals

The present invention also provides a non-human transgenic mammal comprising a functional deletion of EIF5, HNRNPU, BST1, FBXL5, FUT4 or SNORA28, wherein the mammal has decreased susceptibility to infection by a pathogen, such as a virus, a bacterium, a fungus or a parasite. Exemplary transgenic non-human mammals include, but are not limited to, ferrets, fish, guinea pigs, chinchilla, mice, monkeys, rabbits, rats, chickens, cows, and pigs. Such knock-out animals are useful for reducing the transmission of viruses from animals to humans and for further validating a target. In the transgenic animals of the present invention one or both alleles of EIF5, HNRNPU, BST1, FBXL5, FUT4 or SNORA28 can be functionally deleted.

The present invention also provides a non-human transgenic mammal comprising a functional deletion of a gene set forth in Table 1 wherein the mammal has decreased susceptibility to infection by two or more, three or more, four or more, or five or more pathogens selected from the group consisting of a picornavirus, an orthomyxovirus, a paramyxovirus, a coronavirus, an adenovirus, a flavivirus, a filovirus, a calicivirus or a reovirus. The two or more, three or more, four or more; or five or more pathogens can be respiratory viruses selected from the group consisting of Franciscella tularensis, influenza, RSV, rhinovirus, parainfluenza virus, pox virus, and measles. The two or more, three or more, four or more; or five or more pathogens can be gastrointestinal viruses selected from the group consisting of a reovirus, a Norwalk virus, an Ebola virus, a Marburg virus, a Dengue fever virus, a West Nile virus, a yellow fever virus, a rotavirus and an enterovirus. The two or more, three or more, four or more; or five or more pathogens can be selected from the group consisting of Franciscella tularensis, HIV, a pox virus, a herpes virus, an RSV virus, an influenza virus, a hepatitis C virus, a hepatitis B virus, Epstein Barr Virus, Human Papilloma Virus, CMV, West Nile virus, a rhinovirus, an adenovirus, measles virus, Marburg virus, Ebola virus, Rift Valley Fever Virus, LCM, Junin virus, Machupo virus, Guanarito virus, Lassa Fever virus, Hantavirus, SARS virus, Nipah virus, Caliciviruses, Hepatitis A, LaCrosse, California encephalitis, VEE, EEE, WEE, Japanese Encephalitis Virus, Kyasanur Forest Virus, BVDV, Yellow Fever, Rabies, Chikungunya virus or a Dengue fever virus.

By “decreased susceptibility” is meant that the animal is less susceptible to infection or experiences decreased infection by a pathogen as compared to an animal that does not have one or both alleles of an EIF5, HNRNPU, BST1, FBXL5, FUT4 or SNORA28 functionally deleted. The animal does not have to be completely resistant to the pathogen. For example, the animal can be 10%, 20%, 30%, 40%, 50%, 60%, 70%, 80%, 90%, 100% or any percentage in between less susceptible to infection by a pathogen as compared to an animal that does not have a functional deletion of EIF5, HNRNPU, BST1, FBXL5, FUT4 or SNORA28. Furthermore, decreasing infection or decreasing susceptibility to infection includes decreasing entry, replication, pathogenesis, insertion, lysis, or other steps in the replication strategy of a virus or other pathogen into a cell or subject, or combinations thereof.

Therefore, the present invention provides a non-human transgenic mammal comprising a functional deletion of EIF5, HNRNPU, BST1, FBXL5, FUT4 or SNORA28, wherein the mammal has decreased susceptibility to infection by a pathogen, such as a virus, a bacterium, a parasite or a fungus. A functional deletion is a mutation, partial or complete deletion, insertion, or other variation made to a gene sequence that inhibits production of the gene product or renders a gene product that is not completely functional or non-functional. Functional deletions can be made by insertional mutagenesis (for example via insertion of a transposon or insertional vector), by site directed mutagenesis, via chemical mutagenesis, via radiation or any other method now known or developed in the future that results in a transgenic animal with a functional deletion of EIF5, HNRNPU, BST1, FBXL5, FUT4 or SNORA28.

Alternatively, a nucleic acid sequence such as siRNA, a morpholino or another agent that interferes with EIF5, HNRNPU, BST1, FBXL5, FUT4 or SNORA28 mRNA expression can be delivered. The expression of the sequence used to knock-out or functionally delete the desired gene can be regulated by an appropriate promoter sequence. For example, constitutive promoters can be used to ensure that the functionally deleted gene is not expressed by the animal. In contrast, an inducible promoter can be used to control when the transgenic animal does or does not express the gene of interest Exemplary inducible promoters include tissue-specific promoters and promoters responsive or unresponsive to a particular stimulus (such as light, oxygen, chemical concentration, such as a tetracycline inducible promoter).

The transgenic animals of the present invention that comprise a functionally deleted EIF5, HNRNPU, BST1, FBXL5, FUT4 or SNORA28 gene can be examined during exposure to various pathogens. Comparison data can provide insight into the life cycles of pathogens. Moreover, knock-out animals or functionally deleted (such as birds or pigs) that are otherwise susceptible to an infection (for example influenza) can be made to resist infection, conferred by disruption of the gene. If disruption of the gene in the transgenic animal results in an increased resistance to infection, these transgenic animals can be bred to establish flocks or herds that are less susceptible to infection.

Transgenic animals, including methods of making and using transgenic animals, are described in various patents and publications, such as WO 01/43540; WO 02/19811; U.S. Pub. Nos: 2001-0044937 and 2002-0066117; and U.S. Pat. Nos. 5,859,308; 6,281,408; and 6,376,743; and the references cited therein.

The transgenic animals of this invention also include conditional gene knockdown animals produced, for example, by utilizing the SIRIUS-Cre system that combines siRNA for specific gene-knockdown, Cre-loxP for tissue-specific expression and tetracycline-on for inducible expression. These animals can be generated by mating two parental lines that contain a specific siRNA of interest gene and tissue-specific recombinase under tetracycline control. See Chang et al. “Using siRNA Technique to Generate Transgenic Animals with Spatiotemporal and Conditional Gene Knockdown.” American Journal of Pathology 165: 1535-1541 (2004) which is hereby incorporated in its entirety by this reference regarding production of conditional gene knockdown animals.

The present invention also provides cells including an altered or disrupted EIF5, HNRNPU, BST1, FBXL5, FUT4 or SNORA28 that are resistant to infection by a pathogen. These cells can be in vitro, ex vivo or in vivo cells and can have one or both alleles altered. These cells can also be obtained from the transgenic animals of the present invention. Such cells therefore include cells having decreased susceptibility to a virus or any of the other pathogens described herein, including bacteria, parasites and fungi.

Since the genes set forth herein are involved in viral infection, also provided herein are methods of overexpressing any of the genes set forth in Table 1 in host cells. Overexpression of these genes can provide cells that increase the amount of virus produced by the cell, thus allowing more efficient production of viruses. Also provided is the overexpression of the genes set forth herein in avian eggs, for example, in chicken eggs.

Methods of screening agents, such as a chemical, a compound, a small or large molecule, an organic molecule, an inorganic molecule, a peptide, a drug, a protein, a cDNA, an antibody, a morpholino, a triple helix molecule, an siRNA, an shRNAs, an miRNA, an antisense nucleic acid, an LNA or a ribozyme set forth using the transgenic animals described herein are also provided.

### Screening for Resistance to Infection

Also provided herein are methods of screening host subjects for resistance to infection by characterizing a nucleotide sequence of a host EIF5, HNRNPU, BST1, FBXL5, FUT4 or SNORA28 nucleic acid or corresponding amino acid sequence. The nucleic acid or amino acid sequence of a subject can be isolated, sequenced, and compared to the wildtype sequence for EIF5, HNRNPU, BST1, FBXL5, FUT4 or SNORA28. The greater the similarity between that subject's EIF5, HNRNPU, BST1, FBXL5, FUT4 or SNORA28 nucleic acid and the wildtype sequence, the more susceptible that person is to infection, while a decrease in similarity between that subject's EIF5, HNRNPU, BST1, FBXL5, FUT4 or SNORA28 nucleic acid and the wildtype sequence, the more resistant that subject can be to infection. Such screens can be performed for any EIF5, HNRNPU, BST1, FBXL5, FUT4 or SNORA28 host nucleic acid or the corresponding amino acid sequence in any species.

Assessing the genetic characteristics of a population can provide information about the susceptibility or resistance of that population to bacterial or viral infection. For example, polymorphic analysis of alleles in a particular human population, such as the population of a particular city or geographic area, can indicate how susceptible that population is to infection. A higher percentage of alleles substantially similar to wildtype EIF5, HNRNPU, BST1, FBXL5, FUT4 or SNORA28 can indicate that the population is more susceptible to infection, while a large number of polymorphic alleles that are substantially different than wild-type EIF5, HNRNPU, BST1, FBXL5, FUT4 or SNORA28 sequences can indicate that a population is more resistant to infection. Such information can be used, for example, in making public health decisions about vaccinating susceptible populations.

The present invention also provides a method of screening a cell for a variant form of EIF5, HNRNPU, BST1, FBXL5, FUT4 or SNORA28. A variant can be a gene with a functional deletion, mutation or alteration in the gene such that the amount or activity of the gene product is altered. These cells containing a variant form of a gene can be contacted with a pathogen to determine if cells comprising a naturally occurring variant of EIF5, HNRNPU, BST1, FBXL5, FUT4 or SNORA28 differ in their resistance to infection. For example, cells from an animal, for example, a chicken, can be screened for a variant form of EIF5, HNRNPU, BST1, FBXL5, FUT4 or SNORA28. If a naturally occurring variant is found and chickens possessing a variant form of the gene in their genome are less susceptible to infection, these chickens can be selectively bred to establish flocks that are resistant to infection. By utilizing these methods, flocks of chickens that are resistant to avian flu or other pathogens can be established. Similarly, other animals can be screened for a variant form of a gene EIF5, HNRNPU, BST1, FBXL5, FUT4 or SNORA28. If a naturally occurring variant is found and animals possessing a variant form of the gene in their genome are less susceptible to infection, these animals can be selectively bred to establish populations that are resistant to infection. These animals include, but are not limited to, cats, dogs, livestock (for example, cattle, horses, pigs, sheep, goats, etc.), laboratory animals (for example, mouse, monkey, rabbit, rat, gerbil, guinea pig, etc.) and avian species (for example, flocks of chickens, geese. turkeys, ducks, pheasants, pigeons, doves etc.). Therefore, the present application provides populations of animals that comprise a naturally occurring variant of EIF5, HNRNPU, BST1, FBXL5, FUT4 or SNORA28 that results in decreased susceptibility to viral infection, thus providing populations of animals that are less susceptible to viral infection. Similarly, if a naturally occurring variant is found and animals possessing a variant form of the gene in their genome are less susceptible to bacterial, parasitic or fungal infection, these animals can be selectively bred to establish populations that are resistant to bacterial, parasitic or fungal infection.

Also provided is a method of making a compound that decreases infection of a cell by a pathogen, comprising: a) synthesizing a compound; b) administering the compound to a cell containing a cellular gene encoding EIF5, HNRNPU, BST1, FBXL5, FUT4 or SNORA28; c) contacting the cell with an infectious pathogen; d) determining the level of infection, a decrease or elimination of infection indicating that the agent is an agent that decreases infection; e) associating the agent with decreasing expression or activity of EIF5, HNRNPU, BST1, FBXL5, FUT4 or SNORA28.

This method can further comprise making the association by measuring the level of expression and/or activity of EIF5, HNRNPU, BST1, FBXL5, FUT4 or SNORA28.

Further provided is a method of making a compound that decreases infection in a cell by a pathogen, comprising: a) optimizing a compound to bind EIF5, HNRNPU, BST1, FBXL5, FUT4 or SNORA28 or gene product of EIF5, HNRNPU, BST1, FBXL5, FUT4 or SNORA28; b) administering the compound to a cell containing a cellular gene encoding EIF5, HNRNPU, BST1, FBXL5, FUT4 or SNORA28; c) contacting the cell with an infectious pathogen; d) determining the level of infection, a decrease or elimination of infection indicating the making of a compound that decreases infection in a cell by a pathogen. This method can further comprise making a compound that decreases infection in a cell by a pathogen comprising synthesizing therapeutic quantities of the compound made.

The present invention also provides a method of synthesizing a compound that binds to a gene product of EIF5, HNRNPU, BST1, FBXL5, FUT4 or SNORA28 and decreases infection by a pathogen comprising: a) contacting a library of compounds with a gene product of EIF5, HNRNPU, BST1, FBXL5, FUT4 or SNORA28; b) associating binding with a decrease in infection; and c) synthesizing derivatives of the compounds from the library that bind to the gene product of EIF5, HNRNPU, BST1, FBXL5, FUT4 or SNORA28.

Further provided is a business method to reduce the cost of discovery of drugs that can reduce infection by a pathogen comprising: a) screening, outside of the United States, for drugs that reduce infection by binding to or reducing the function of a gene product of EIF5, HNRNPU, BST1, FBXL5, FUT4 or SNORA28; and b) importing active drugs into the United States.

Also provided is a method of making drugs comprising directing the synthesis of drugs that reduce infection by binding to or reducing the function of EIF5, HNRNPU, BST1, FBXL5, FUT4 or SNORA28 or gene product of EIF5, HNRNPU, BST1, FBXL5, FUT4 or SNORA28.

The following examples are put forth so as to provide those of ordinary skill in the art with a complete disclosure and description of how the antibodies, polypeptides, nucleic acids, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### EXAMPLES

Following infection with the U3NeoSV1 retrovirus gene trap shuttle vector, libraries of mutagenized Vero cells were isolated in which each clone contained a single gene disrupted by provirus integration. Gene entrapment was performed essentially as described in U.S. Pat. No. 6,448,000 and U.S. Pat. No. 6,777,177. The entrapment libraries were infected with Franciscella tularensis and resistant clones were selected as described below.

Four days prior to infection, THP1 gene trap library cells (human acute monocytic leukemia cell line) were thawed at room temperature. 13 mLs of complete growth medium and a thawed gene trap library aliquot were combined in a sterile 15 mL conical tube. This was centrifuged at 1000 rpm for 5 minutes to pellet the cells. The supernatant was discarded and the cells were resuspended in complete growth medium and the aliquot of cells seeded into 4 T150 flask. The cells were allowed to grow for 4 days at 37° C. in 5% CO₂ or until the cells were 70-100% confluent. On the day of infection, the medium in the T150 flasks was replaced with 19 mLs of fresh complete growth medium immediately before infecting the cells. Franciscella tularensis was thawed from the −80° C. freezer at 4° C. for 30 minutes and was diluted in complete growth medium. 1 mL of diluted virus was added to each of the 4 T150 flasks containing THP1 gene trap library cells. The cells were incubated at 37° C., 5% CO₂ for 2 hours. The medium was discarded from the flasks into the waste container and replaced with 20 mLs of fresh complete growth medium to remove the inoculum. The cells were incubated at 37° C., 5% CO₂. Infection was allowed to proceed without changing the medium until the cells were approximately 90% dead or dying (routinely 3 or 4 days post-infection). From then on, the medium was changed daily through day 7 post-infection. The medium was changed on days 10, 14, 17, 21, etc. post-infection. Resistant colonies (clones) were observed 2-3 weeks post-infection by examining the under side of the flasks. Resistant cells were trypsinized and cells from each resistant clone were transferred to a single well of a24 well plate (already containing 1 ml of complete growth medium). This process was repeated for each colony. The colonies were grown until cells in several wells approach, 20-30% confluency. At this point, cells were detached and seeded into duplicate 24-well plates. Resistance confirmation was performed by re-infecting clones in one 24-well plate. Following identification of resistant clones, resistant clones in the uninfected 24-well plates were expanded in T75 flasks for subsequent genomic DNA isolation (DNeasy kits, Qiagen, Inc.).

### Identification of Genes Disrupted in Tularemia Resistant Clones

The U3NeoSV1 gene trap vector contains a plasmid origin of replication and ampicillin resistance gene; thus, regions of genomic DNA adjacent to the targeting vector were readily cloned by plasmid rescue and sequenced. The flanking sequences were compared to the nucleic acid databases to identify candidate cellular genes that confer resistance to lytic infection by franscicella tularensis when altered by gene entrapment. These genes are listed in Table 1.

siRNA studies

Cell Lines and Viruses:

Human hepatocellular carcinoma cell lines HepG2 and Hep3B (ATCC) were maintained at 37° C. under 5% CO₂ in Minimum Essential Medium Eagle (Mediatech) supplemented with 5% fetal bovine serum, 2 mM L-glutamine, 100 U/ml penicillin and 100 ug/ml streptomycin. African green monkey kidney Vero E6 cells supplied by ATCC or Vanderbilt University, and the cervical carcinoma derived TZM-bl cell line (NIH AIDS Reasearch and Reference Reagent Program) were maintained at 37° C. under 5% CO₂ in Dulbecco's Modified Eagle Medium (Mediatech) supplemented with 5% fetal bovine serum, 2 mM L-glutamine, 100 U/ml penicillin and 100 ug/ml streptomycin.

Influenza A strain 1520 (ATCC) was grown in HepG2 cells. Dengue was grown in Hep3B cells. Herpes simplex virus strain 186, respiratory syncytial virus and cowpox virus were grown in Vero E6 cells, Human rhinovirus-16 was grown in TZM-bl cells cultured at 32° C.

siRNA Transfections:

Pools of 4 duplexed siRNA molecules targeting a gene product set forth in Table 1 were reconstituted to a final working concentration of 50 uM as directed by the manufacturer. Working volume aliquots were frozen at −20° C. to reduce or eliminate freeze'thaw cycles. Twenty-four hours prior to transfection, cells were plated in 6-well dishes at 3×10⁵ cells per well, such that at the time of transfection, the cells would be at approximately 30% confluence. Prior to transfection, the cells were washed once with 1× phosphate buffered saline, and the medium replaced with approximately 1.8 ml antibiotic-free medium. siRNA aliquots were diluted with Opti-MEM and RNAseOUT (Invitrogen), 100 ul and 1 ul per transfection, respectively. In a separate tube, transfection reagent Lipofectamine-2000 (Invitrogen) for transfections into HepG2 or Vero E6 cellsor Oligofectamine (Invitrogen) for transfections into Hep3B cells was diluted in Opti-MEM as directed by the manufacturer. Following a 5 minute incubation at room temperature, the diluted siRNA was added to the transfection reagent mixture, and was incubated for an additional 20 minutes prior to adding to independent wells of the 6-well dishes. In contrast, HiPerFect (QIAGEN) was added directly to siRNA dilutions for transfections into TZM-bl cells as directed by the manufacturer. Transfections were incubated at 37° C. for 48 hours without changing the medium.

Virus Infections:

Following 48-hour transfection, medium was aspirated from 6-well plates. Viruses were diluted in the appropriate medium and 500 ul of either virus-free medium or virus dilution was added to each well, and adsorption was allowed to occur at the appropriate temperature for 1 hour. Following adsorption, inoculum was aspirated off the cells, cells were washed once with 1× phosphate buffered saline, and 2 ml growth medium was added to the cells. The infected cells were incubated for 72 hours at the appropriate temperature prior to harvesting samples for viral titration.

Viral Genomic Extractions:

Seventy-hours after inoculating cells, medium was harvested from 6-well dishes and centrifuged for 2 minutes at 10,000 rpm to remove any cellular debris. 200 ul of clarified medium was added to 25 ul Proteinase K, to which 200 ul PureLink96 Viral RNA/DNA lysis buffer (Invitrogen) was added according to the manufacturer. Samples were processed and viral genomic RNA or DNA was extracted using an epMotion 5075 robotics station (Eppendorf) and the PureLink96 Viral RNA/DNA kit (Invitrogen).

cDNA and Quantitative Real-Time PCR Reactions:

3 ul of extracted viral RNA was converted to cDNA using M-MLV reverse transcriptase (Invitrogen) and AmpliTaq Gold PCR buffer (Applied Biosystems). MgCl₂, dNTPs and RNAseOUT (Invitrogen) were added to achieve a final concentration of 5 mM, 1 mM and 2 U/ul, respectively. Random hexamers (Applied Biosystems) were added to obtain 2.5 mM final concentration. Reactions were incubated at 42° C. for 1 hour, followed by heat inactivation of the reverse transcriptase at 92° C. for 10 minutes. Quantitative real-time PCR reactions were set up in 10 ul volumes using 1 ul of template cDNA or extracted viral DNA using virus-specific TaqMan probes (Applied Biosystems) and RealMasterMix (Eppendorf). 2-step reactions were allowed to proceed through 40 to 50 cycles on an ep RealPlex thermocycler (Eppendorf). Quantitative standards for real-time PCR were constructed by cloning purified amplicons into pCR2-TOPO (Invitrogen) and sequenced as necessary.

Table 3 shows gene products set forth in Table 1 for which at least 50% inhibition of viral replication (influenza, RSV, HSV, polio, cowpox, rhinovirus (R-16) or Dengue) was observed via siRNA experiments The siRNAs utilized in these experiments are set forth above.

Throughout this application, various publications are referenced. The disclosures of these publications in their entireties are hereby incorporated by reference into this application in order to more fully derive the state of the art to which this invention pertains.

What is claimed is:

1. A method of decreasing infection in a cell by a pathogen comprising decreasing expression or activity of E1F5, HNRNPU, BST1, FBXL5, FUT4 or SNORA28.

2. The method of claim 1, wherein infection is decreased by decreasing the replication of the pathogen.

3-16. (canceled)

17. A method of decreasing infection in a subject comprising administering to the subject an effective amount of a composition that decreases expression or activity of E1F5, HNRNPU, BST1, FBXL5, FUT4 or SNORA28, wherein the composition inhibits infection by two or more-respiratory viruses selected from the group consisting of: a picornavirus, an orthomyxovirus, a paramyxovirus, a coronavirus and an adenovirus.

18. The method of claim 17, wherein the two or more respiratory viruses are selected from the group consisting of an influenza virus, a pox virus, parainfluenza virus, adenovirus, measles, rhinovirus and RSV.

19-23. (canceled)

24. A non-human transgenic mammal comprising a functional deletion of E1F5, HNRNPU, BST1, FBXL5, FUT4 or SNORA28, wherein the mammal has decreased susceptibility to infection by a pathogen.

25. The transgenic mammal of claim 24, wherein one or both alleles are functionally deleted to decrease infection.

26-49. (canceled)
